(19) United States
(12) Patent Application Publication
(10) Pub. No.: US 2025/0387006 A1
(43) Pub. Date: Dec. 25, 2025

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Rito MURASE, Tokyo (JP)

(73) Assignee: FUJIFILM Corporation, Tokyo (JP)

(21) Appl. No.: 19/214,021

(22) Filed: May 20, 2025

(30) Foreign Application Priority Data
Jun. 19, 2024 (JP) 2024-099032

(51) Int. Cl. A61B 1/00; G06T 7/00

(52) U.S. Cl. CPC A61B 1/000094; A61B 1/0005; G06T 7/0012; G06T 2207/10068; G06T 2207/20084; G06T 2207/30004

(57) ABSTRACT

A medical support device includes a processor. The processor is configured to input a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions. In addition, the processor is configured to output lumen specification information for specifying a lumen existence region in which an existence position of the lumen is specified with higher accuracy than in the divided regions in the medical image or the image corresponding to the medical image, based on the plurality of divided regions and the plurality of confidence levels.

### CROSS-REFERENCE TO RELATED APPLICATIONS

This application claims priority under 35 USC 119 from Japanese Patent Application No. 2024-099032 filed on Jun. 19, 2024, the disclosure of which is incorporated by reference herein.

### BACKGROUND

### 1. Technical Field

The present disclosure relates to a medical support device, an endoscope system, a medical support method, and a program.

### 2. Description of the Related Art

JP2003-093328A discloses an endoscope insertion direction detection method comprising a first step of inputting an endoscopic image, a second step of detecting a direction of brightness variation in the endoscopic image, and a third step of generating information on an insertion direction of an endoscope based on a result of the detection. In addition, JP2003-093328A also discloses an endoscope insertion direction detection method comprising a first step of setting insertion candidate directions that are candidates for an insertion direction of an endoscope, a second step of inputting an endoscopic image, a third step of detecting a direction of brightness variation in the endoscopic image, a fourth step of evaluating a similarity among a plurality of insertion candidate directions and the direction of brightness variation, and a fifth step of determining the insertion direction of the endoscope based on a result of the evaluation.

WO2020/194472A discloses a movement support system comprising a multiple-operation information calculation unit that calculates multiple-operation information indicating a plurality of operations, which are different in time and correspond to a multiple-operation target scene that is a scene requiring a plurality of operations different in time, based on a captured image acquired by an imaging unit disposed in an insertion part, and a presentation information generation unit that generates presentation information for the insertion part based on the multiple-operation information calculated by the multiple-operation information calculation unit.

### SUMMARY

An embodiment according to the present disclosure provides a medical support device, an endoscope system, a medical support method, and a program that enable a user or the like to ascertain a position of a lumen, which is shown in a medical image, in the medical image with high accuracy.

A first aspect according to the present disclosure relates to a medical support device comprising: a processor, in which the processor is configured to: input a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and output lumen specification information for specifying a lumen existence region in which an existence position of the lumen is specified with higher accuracy than in the divided regions in the medical image or the image corresponding to the medical image, based on the plurality of divided regions and the plurality of confidence levels.

A second aspect according to the present disclosure relates to the medical support device according to the first aspect, in which the lumen existence region is a region in which a position at which the lumen is shown in the medical image is specifiable with a higher resolution than in the plurality of divided regions, along the circumferential direction.

A third aspect according to the present disclosure relates to the medical support device according to the first or second aspect, in which a direction from a reference position of the medical image or the image corresponding to the medical image to an existence position of each of the plurality of divided regions is determined by a plurality of first vectors, a direction from the reference position to the lumen existence region is determined by a second vector, the second vector is a sum of at least two third vectors obtained by adding the confidence level, as a weight, to at least two first vectors among the plurality of first vectors, and the lumen specification information is information determined based on the second vector.

A fourth aspect according to the present disclosure relates to the medical support device according to any one of the first to third aspects, in which the processor is configured to output the medical image or the image corresponding to the medical image, and the lumen specification information is updated in accordance with an output timing of the medical image or the image corresponding to the medical image.

A fifth aspect according to the present disclosure relates to the medical support device according to any one of the first to fourth aspects, in which the output of the lumen specification information is implemented by displaying the lumen specification information on a screen.

A sixth aspect according to the present disclosure relates to the medical support device according to the fifth aspect, in which the medical image or the image corresponding to the medical image is displayed on the screen, and the lumen specification information displayed on the screen is updated in accordance with a display timing of the medical image or the image corresponding to the medical image.

A seventh aspect according to the present disclosure relates to the medical support device according to the fifth aspect or the sixth aspect, in which the medical image and/or the image corresponding to the medical image and the lumen specification information are displayed on the screen in a comparable manner.

An eighth aspect according to the present disclosure relates to the medical support device according to the seventh aspect, in which the lumen specification information is displayed in a superimposed manner on the medical image and/or the image corresponding to the medical image.

A ninth aspect according to the present disclosure relates to the medical support device according to any one of the first to eighth aspects, in which a direction from a reference position of the medical image or the image corresponding to the medical image to an existence position of each of the plurality of divided regions is determined by a plurality of first vectors, a direction from the reference position to the lumen existence region is determined by a second vector, the second vector is a sum of at least two third vectors obtained by adding the confidence level, as a weight, to at least two first vectors among the plurality of first vectors, and the lumen specification information includes a mark for specifying a region determined as the lumen existence region based on the second vector in the medical image or the image corresponding to the medical image.

A tenth aspect according to the present disclosure relates to the medical support device according to the ninth aspect, in which a shape of the mark is an arc, and a center of the arc is a center of the medical image or the image corresponding to the medical image.

An eleventh aspect according to the present disclosure relates to the medical support device according to the ninth aspect, in which a shape of the mark is a shape along an outer edge of the medical image or an outer edge of the image corresponding to the medical image.

A twelfth aspect according to the present disclosure relates to the medical support device according to any one of the ninth to eleventh aspects, in which a plurality of markers that are hidden are associated with the medical image or the image corresponding to the medical image, and the mark is displayed on the screen by displaying at least one marker corresponding to a position of the lumen existence region among the plurality of markers.

A thirteenth aspect according to the present disclosure relates to an endoscope system comprising: the medical support device according to any one of the first to twelfth aspects; and an endoscope, in which the medical image is generated by imaging the inside of the luminal organ including the lumen with the endoscope.

A fourteenth aspect according to the present disclosure relates to a medical support method comprising: inputting a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and outputting lumen specification information for specifying a lumen existence region in which an existence position of the lumen is specified with higher accuracy than in the divided regions in the medical image or the image corresponding to the medical image, based on the plurality of divided regions and the plurality of confidence levels.

A fifteenth aspect according to the present disclosure relates to a program causing a computer to execute a process comprising: inputting a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and outputting lumen specification information for specifying a lumen existence region in which an existence position of the lumen is specified with higher accuracy than in the divided regions in the medical image or the image corresponding to the medical image, based on the plurality of divided regions and the plurality of confidence levels.

### BRIEF DESCRIPTION OF THE DRAWINGS

Exemplary embodiments of the technology of the disclosure will be described in detail based on the following figures, wherein:

FIG. 1 is a conceptual diagram showing an aspect example in which an endoscope system is used by a doctor;

FIG. 2 is a conceptual diagram showing an example of an overall configuration of the endoscope system;

FIG. 3 is a block diagram showing an example of a hardware configuration of an electrical system of the endoscope system;

FIG. 4 is a block diagram showing an example of main functions of a processor included in a medical support device and an example of information stored in a storage;

FIG. 5 is a block diagram showing an example of a hardware configuration of an electrical system of an information processing device;

FIG. 6 is a conceptual diagram showing an aspect example in which training data is generated by the information processing device;

FIG. 7 is a conceptual diagram showing an example of an example image;

FIG. 8 is a conceptual diagram showing an example of training data generated in a case in which a lumen is shown in a region other than a central region of the example image shown in FIG. 7;

FIG. 9 is a conceptual diagram showing an example of training data generated in a case in which the lumen is shown in the central region of the example image shown in FIG. 7;

FIG. 10 is a conceptual diagram showing an example of processing contents in the information processing device in a case in which a lumen recognition model is generated by training a model through machine learning using training data;

FIG. 11 is a conceptual diagram showing an example of processing contents of a recognition unit of the medical support device;

FIG. 12 is a conceptual diagram showing an example of confidence level information generated by the lumen recognition model in a case in which the lumen is shown in a frame;

FIG. 13 is a conceptual diagram showing an example of a relationship among a plurality of divided regions obtained by radially dividing a map included in the confidence level information and a plurality of direction unit vectors added to the plurality of divided regions;

FIG. 14 is a conceptual diagram showing an example of processing contents of a controller of the medical support device;

FIG. 15 is a conceptual diagram showing an aspect example in which, in a case in which the lumen is shown in a region other than a central region of the frame, the frame is displayed in a first display region of a screen, a mark for specifying a position of a lumen existence region, in which an existence position of the lumen shown in the frame is specified, in the frame is displayed in a superimposed manner on the frame, and visible information is displayed in a second display region of the screen as one piece of auxiliary information;

FIG. 16 is a conceptual diagram showing an aspect example in which, in a case in which the lumen is shown in the central region of the frame, the frame is displayed in the first display region of the screen, the mark for specifying the position of the lumen existence region, in which the existence position of the lumen shown in the frame is specified, in the frame is displayed in a superimposed manner on the frame, and the visible information is displayed in the second display region of the screen as one piece of the auxiliary information;

FIG. 17 is a flowchart showing an example of a flow of machine learning processing;

FIG. 18 is a flowchart showing an example of a flow of medical support processing;

FIG. 19 is a conceptual diagram showing a form example in which a mark is displayed along an outer edge of the frame displayed on a screen;

FIG. 20 is a conceptual diagram showing a form example in which the mark is displayed in a superimposed manner on the frame by displaying at least one marker corresponding to the position of the lumen existence region among a plurality of hidden markers associated with the map and/or the frame;

FIG. 21 is a conceptual diagram showing a form example in which an outer contour line that is a line that outlines an outer contour of the lumen existence region is displayed in a superimposed manner on the frame; and

FIG. 22 is a conceptual diagram showing an example of a series of processing in which a processor included in a computer issues a processing execution request to an external device via a network, the external device executes processing in response to the processing execution request, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings. It should be noted that the present disclosure can also be applied to a program and a computer program product.

First, the terms used in the following description will be described.

CPU is an abbreviation for “central processing unit”. GPU is an abbreviation for “graphics processing unit”. GPGPU is an abbreviation for “general-purpose computing on graphics processing units”. APU is an abbreviation for “Accelerated Processing Unit”. TPU is an abbreviation for “Tensor Processing Unit”. RAM is an abbreviation for “random-access memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. LAN is an abbreviation for “local area network”. WAN is an abbreviation for “wide area network”. 5G is an abbreviation for “5th generation mobile communication system”.

In the following description, a processor with a reference numeral (hereinafter, simply referred to as a “processor”) may be one computing device or may be a combination of a plurality of computing devices. The processor may be one type of computing device or may be a combination of a plurality of types of computing devices. Examples of the computing device include a CPU, a GPU, a GPGPU, an APU, and a TPU.

In the following description, a memory with a reference numeral is a memory, such as a RAM, that temporarily stores information, and is used by the processor as a work memory.

In the following description, a storage with a reference numeral is one or a plurality of non-volatile storage devices that store various programs, various parameters, and the like. Examples of the non-volatile storage device include a flash memory, a magnetic disk, and a magnetic tape. Another example of the storage is a cloud storage.

In the embodiment described below, an external I/F with a reference numeral controls the transmission and reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi®, and Bluetooth®.

In the embodiment described below, “A and/or B” is synonymous with “at least one of A or B”. That is, “A and/or B” may mean only A, may mean only B, or may mean a combination of A and B. In addition, in the present specification, the same concept as “A and/or B” is applied to a case in which the connection of three or more matters is expressed by “and/or”.

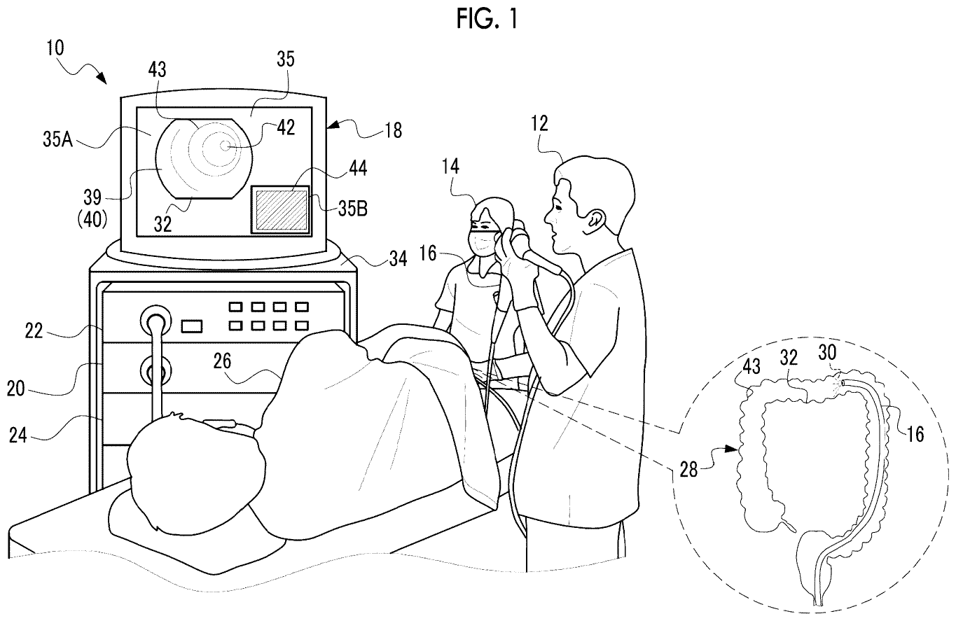

FIG. 1 is a conceptual diagram showing an aspect example in which an endoscope system 10 is used. As shown in FIG. 1, the endoscope system 10 is used by a doctor 12 in an endoscopy and the like. A staff member 14, such as a nurse, assists with the endoscopy.

The endoscope system 10 is connected to a communication device (not shown) in a communicable manner, and information obtained by the endoscope system 10 is transmitted to the communication device. Examples of the communication device include a server, a personal computer, and/or a tablet terminal that manage various types of information, such as electronic medical records. The communication device receives the information transmitted from the endoscope system 10 and executes processing using the received information (for example, processing of storing the information in the electronic medical record or the like).

The endoscope system 10 comprises an endoscope 16, a display device 18, a light source device 20, a control device 22, and a medical support device 24. In the present embodiment, the endoscope system 10 is an example of an “endoscope system” according to the present disclosure, the endoscope 16 is an example of an “endoscope” according to the present disclosure, and the medical support device 24 is an example of a “medical support device” according to the present disclosure.

The endoscope system 10 is a modality for performing a medical examination on a large intestine 28, which is a luminal organ included in a body of a subject 26 (for example, a patient), by using the endoscope 16. In the present embodiment, the large intestine 28 is an object that is observed by the doctor 12.

The endoscope 16 is used by the doctor 12, and is inserted into the body of the subject 26. In the present embodiment, the endoscope 16 is inserted into the large intestine 28 of the subject 26. In the present embodiment, the large intestine 28 is an example of a “luminal organ” according to the present disclosure.

The endoscope system 10 images an inside of the large intestine 28 including a lumen 42 by using the endoscope 16 inserted into the large intestine 28 of the subject 26, and performs various medical treatments on the large intestine 28 as necessary. The large intestine 28 has the lumen 42. The endoscope 16 is inserted into the lumen 42. A position of the lumen 42 in the large intestine 28 can be medically specified based on a form pattern of a plurality of folds 43 (for example, a shape, an orientation, and the like of the plurality of folds 43) which are characteristic regions in the large intestine 28. In the present embodiment, as will be described in detail below, the position of the lumen 42 is recognized by AI that has been trained using various types of information, such as the form pattern of the plurality of folds 43, through machine learning, and a result of the recognition is provided as visually ascertainable information to the doctor 12. In the present embodiment, the lumen 42 is an example of a “lumen” according to the present disclosure.

The endoscope system 10 acquires an image showing an aspect including the lumen 42 in the large intestine 28 by imaging the inside of the large intestine 28 including the lumen 42, and outputs the acquired image. In the present embodiment, the endoscope system 10 has an optical imaging function of emitting light 30 in the large intestine 28 and imaging reflected light obtained by being reflected by an intestinal wall 32 of the large intestine 28. It should be noted that, here, the endoscopy of the large intestine 28 has been described as an example, but this is merely an example, and the present disclosure is applicable to an endoscopy of a luminal organ, such as an esophagus, a stomach, a duodenum, or a trachea.

The light source device 20, the control device 22, and the medical support device 24 are installed in a wagon 34. The wagon 34 is provided with a plurality of tables along an up-down direction, and the medical support device 24, the light source device 20, and the control device 22 are installed from a lower table to an upper table. The display device 18 is installed on an uppermost table in the wagon 34.

The control device 22 controls the entire endoscope system 10. The control device 22 executes various types of processing on the image obtained by imaging the intestinal wall 32 with the endoscope 16. In addition, the medical support device 24 executes AI-type processing or the like on the image that has been subjected to various types of processing by the control device 22, under the control of the control device 22, and outputs various types of information including a processing result of the AI-type processing or the like. Examples of an output destination of the various types of information include the display device 18, a stationary storage medium (for example, a storage provided in the endoscope system 10, a storage of a server or the like that is connected to the endoscope system 10 in a communicable manner, and the like), and/or a portable storage medium (for example, a memory card, a USB flash drive, and the like).

The display device 18 displays various types of information (for example, various types of information output from the medical support device 24). Examples of the display device 18 include a liquid-crystal display and an EL display. A tablet terminal with a display may be used instead of the display device 18 or together with the display device 18.

A screen 35 is displayed on the display device 18. The screen 35 includes a plurality of display regions. The plurality of display regions are arranged on the screen 35. In the example shown in FIG. 1, a first display region 35A and a second display region 35B are shown as examples of the plurality of display regions. The first display region 35A has a larger size than the second display region 35B. The first display region 35A is used as a main display region, and the second display region 35B is used as a sub-display region. A size relationship between the first display region 35A and the second display region 35B is not limited to this, and need only be a size relationship that can be included within the screen 35.

An endoscope video image 39 is displayed in the first display region 35A. The endoscope video image 39 is obtained by executing various types of processing on a plurality of images arranged in time series obtained by imaging the inside of the large intestine 28 of the subject 26 with the endoscope 16. The intestinal wall 32 shown in the endoscope video image 39 includes the lumen 42 as a region of interest (that is, an observation target region) at which the doctor 12 gazes, and the doctor 12 can visually recognize the aspect of the intestinal wall 32 including the lumen 42 through the endoscope video image 39.

The image displayed in the first display region 35A is one frame 40 included in a video image including a plurality of frames 40 arranged in time series. That is, the plurality of frames 40 arranged in time series are displayed in the first display region 35A at predetermined frame rates (for example, a dozen frames/second or a few dozen frames/second). In the present embodiment, the frame 40 is an example of a “medical image” according to the present disclosure.

Examples of the video image displayed in the first display region 35A include a video image in a live view mode. The live view mode is merely an example, and the video image may be a video image, such as a video image in a post view mode, that is temporarily stored in a memory or the like and then displayed. In addition, each frame included in a video image for recording stored in the memory or the like may be reproduced and displayed as the endoscope video image 39 on the screen 35 (for example, in the first display region 35A).

The second display region 35B is displayed at the lower right of the screen 35 in a front view. The second display region 35B may be displayed at any position as long as the position is within the screen 35 of the display device 18, but is preferably displayed at a position that is comparable with the endoscope video image 39. Auxiliary information 44 for assistance of the doctor 12 in a medical determination or the like is displayed in the second display region 35B. The auxiliary information 44 is information to be referred to by the doctor 12. Examples of the auxiliary information 44 include various types of information on the subject 26 in which the endoscope 16 is inserted and/or various types of information obtained by executing medical support processing which will be described below.

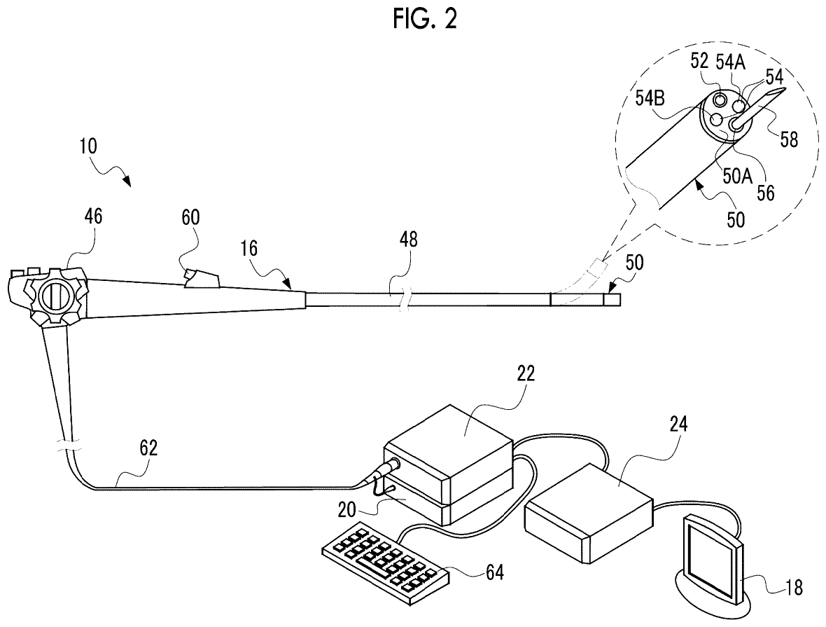

FIG. 2 is a conceptual diagram showing an example of an overall configuration of the endoscope system 10. As shown in FIG. 2, the endoscope 16 comprises an operating part 46 and an insertion part 48. The insertion part 48 is partially curved by the operation of the operating part 46. The insertion part 48 is inserted into the large intestine 28 while being curved along the shape of the large intestine 28 (see FIG. 1) in accordance with the operation of the operating part 46 by the doctor 12 (see FIG. 1).

A camera 52, an illumination device 54, and a treatment tool opening 56 are provided at a distal end portion 50 of the insertion part 48. A portion of the camera 52 (for example, an imaging optical system) and a portion of the illumination device 54 (for example, an irradiation optical system) are exposed from a distal end surface 50A of the distal end portion 50.

The camera 52 is mounted in the endoscope 16 and is inserted into a body cavity of the subject 26 to image the observation target region. Examples of the camera 52 include a CMOS camera. However, this is merely an example, and the camera 52 may be other types of cameras, such as CCD cameras. In the present embodiment, the camera 52 generates an image showing the aspect including the lumen 42 in the large intestine 28 by imaging the inside of the large intestine 28 including the lumen 42. The image generated by the camera 52 is an image of which an outer shape is circular. For example, the image generated by the camera 52 is processed into a shape in which an upper end portion and a lower end portion are masked, by the control device 22. Accordingly, as shown in FIG. 1, an image in which an upper end edge and a lower end edge are linear and a left side edge and a right side edge are arc-shaped is generated as the frame 40.

The illumination device 54 includes illumination windows 54A and 54B. The illumination device 54 emits the light 30 (see FIG. 1) via the illumination windows 54A and 54B. Examples of a type of the light 30 emitted from the illumination device 54 include visible light (for example, white light) and invisible light (for example, near-infrared light). In addition, the illumination device 54 emits special light via the illumination windows 54A and 54B. Examples of the special light include light for BLI and/or light for LCI. The camera 52 images the inside of the large intestine 28 by using an optical method in a state in which the illumination device 54 irradiates the inside of the large intestine 28 with the light 30.

The treatment tool opening 56 is an opening through which a treatment tool 58 protrudes from the distal end portion 50. Further, the treatment tool opening 56 is also used as a suction port for suctioning blood, internal contaminants, and the like and a sending-out port for sending out fluid.

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example shown in FIG. 2, an aspect is shown in which a biopsy needle protrudes from the treatment tool opening 56 as the treatment tool 58. Here, the biopsy needle has been described as an example of the treatment tool 58, but this is merely an example, and the treatment tool 58 may be grasping forceps, a papillotomy knife, a snare, a catheter, a guide wire, a cannula, and/or a biopsy needle with a guide sheath.

The endoscope 16 is connected to the light source device 20 and the control device 22 through a universal cord 62. The medical support device 24 and a reception device 64 are connected to the control device 22. Further, the display device 18 is connected to the medical support device 24. That is, the control device 22 is connected to the display device 18 via the medical support device 24.

It should be noted that, here, since the medical support device 24 is used as an example of an external device for expanding the functions of the control device 22, the form example has been described in which the control device 22 and the display device 18 are indirectly connected to each other via the medical support device 24, but this is merely an example. For example, the display device 18 may be directly connected to the control device 22. In this case, for example, the functions of the medical support device 24 need only be provided in the control device 22, or the control device 22 need only be provided with a function of directing a server (not shown) to execute the same processing as the processing (for example, the medical support processing which will be described below) executed by the medical support device 24, receiving a result of the processing by the server, and using the result.

The reception device 64 receives an instruction from the doctor 12, and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light to supply the light 30 to the illumination device 54 under the control of the control device 22. A light guide is provided in the illumination device 54, and the light 30 supplied from the light source device 20 is emitted from the illumination windows 54A and 54B through the light guide. The control device 22 causes the camera 52 to perform the imaging in a state in which the light 30 is emitted from the illumination windows 54A and 54B. The control device 22 generates the plurality of frames 40 arranged in time series by processing the outer shape of the image obtained by imaging performed by the camera 52 or adjusting an image quality or the like of the image. Then, the control device 22 outputs the endoscope video image 39 including the plurality of generated frames 40 arranged in time series to a predetermined output destination (for example, the medical support device 24).

The medical support device 24 executes various types of processing on the endoscope video image 39 input from the control device 22 to support a medical treatment (here, for example, endoscopy). The medical support device 24 outputs the endoscope video image 39 subjected to various types of processing to a predetermined output destination (for example, the display device 18).

It should be noted that, here, the form example has been described in which the endoscope video image 39 output from the control device 22 is output to the display device 18 via the medical support device 24, but this is merely an example. For example, an aspect may be adopted in which the control device 22 and the display device 18 are connected to each other, and the endoscope video image 39 that has been subjected to various types of processing by the medical support device 24 is displayed on the display device 18 via the control device 22.

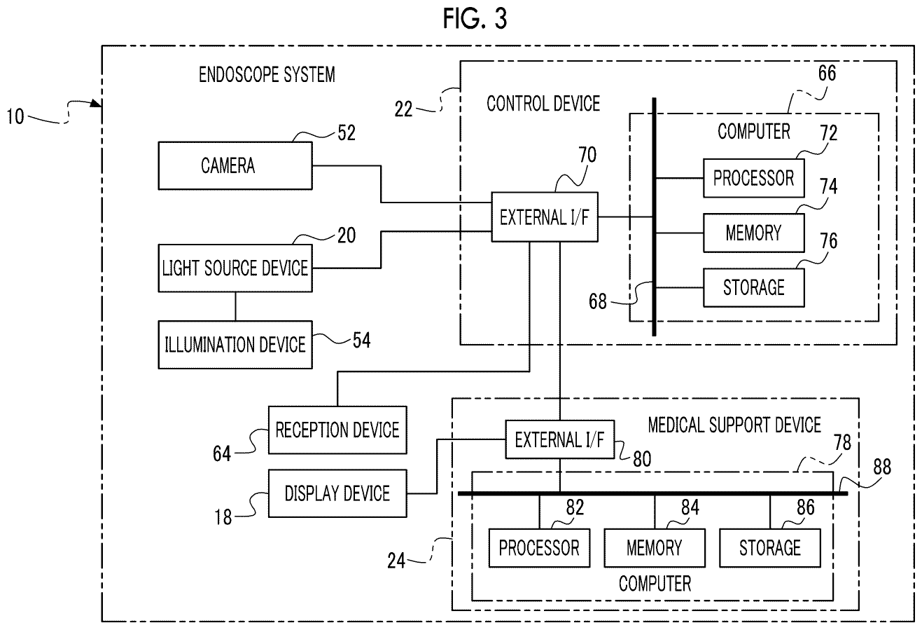

FIG. 3 is a block diagram showing an example of a hardware configuration of an electrical system of the endoscope system 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) existing outside the control device 22 and the processor 72.

The camera 52 is connected to the external I/F 70 as one of the first external devices, and the external I/F 70 transmits and receives various types of information between the camera 52 and the processor 72. The processor 72 controls the camera 52 through the external I/F 70. In addition, the processor 72 generates the endoscope video image 39 (see FIG. 1) by executing various types of processing on the image obtained by imaging the inside of the large intestine 28 (see FIG. 1) with the camera 52, and acquires the generated endoscope video image 39 via the external I/F 70.

The light source device 20 is connected to the external I/F 70 as one of the first external devices, and the external I/F 70 transmits and receives various types of information between the light source device 20 and the processor 72. The light source device 20 supplies light to the illumination device 54 under the control of the processor 72. The illumination device 54 emits the light supplied from the light source device 20.

The reception device 64 is connected to the external I/F 70 as one of the first external devices, and the processor 72 acquires the instruction received by the reception device 64 via the external I/F 70 and executes processing corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a “computer” according to the present disclosure, and the processor 82 is an example of a “processor” according to the present disclosure.

It should be noted that a hardware configuration (that is, the processor 82, the memory 84, and the storage 86) of the computer 78 is essentially the same as the hardware configuration of the computer 66, and thus the description of the hardware configuration of the computer 78 will be omitted here.

The external I/F 80 transmits and receives various types of information between one or more devices (hereinafter, also referred to as “second external devices”) existing outside the medical support device 24 and the processor 82.

The control device 22 is connected to the external I/F 80 as one of the second external devices. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscope video image 39 (see FIG. 1) from the processor 72 of the control device 22 via the external I/Fs 70 and 80, and executes various types of processing on the acquired endoscope video image 39. The various types of processing executed by the processor 82 include AI-type processing (for example, processing using a lumen recognition model 92 described below).

The display device 18 is connected to the external I/F 80 as one of the second external devices. The processor 82 controls the display device 18 via the external I/F 80 such that various types of information (for example, the endoscope video image 39 that has been subjected to various types of processing) are displayed on the display device 18.

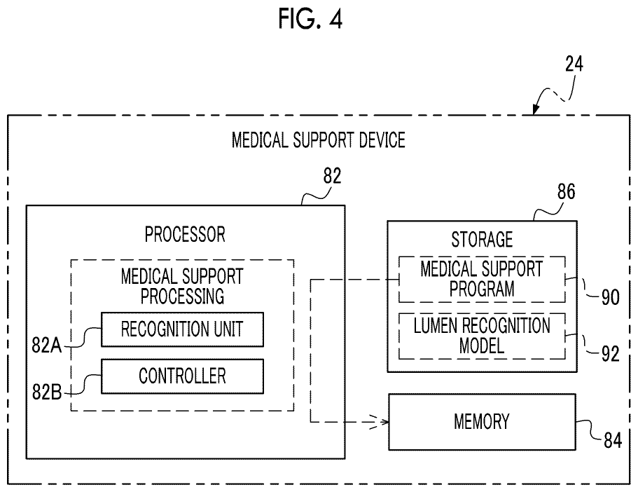

FIG. 4 is a block diagram showing an example of main functions of the processor 82 included in the medical support device 24 and an example of the information stored in the storage 86. As shown in FIG. 4, a medical support program 90 is stored in the storage 86. In the present embodiment, the medical support program 90 is an example of a “program” according to the present disclosure.

The processor 82 reads out the medical support program 90 from the storage 86, and executes the readout medical support program 90 on the memory 84 to execute the medical support processing. The medical support processing is implemented by the processor 82 operating as a recognition unit 82A and a controller 82B in accordance with the medical support program 90 executed on the memory 84.

The lumen recognition model 92 is stored in the storage 86. As will be described in detail below, the lumen recognition model 92 is a trained model that is used in the AI-type processing, and is used by the recognition unit 82A. In the present embodiment, the lumen recognition model 92 is an example of a “trained model” according to the present disclosure.

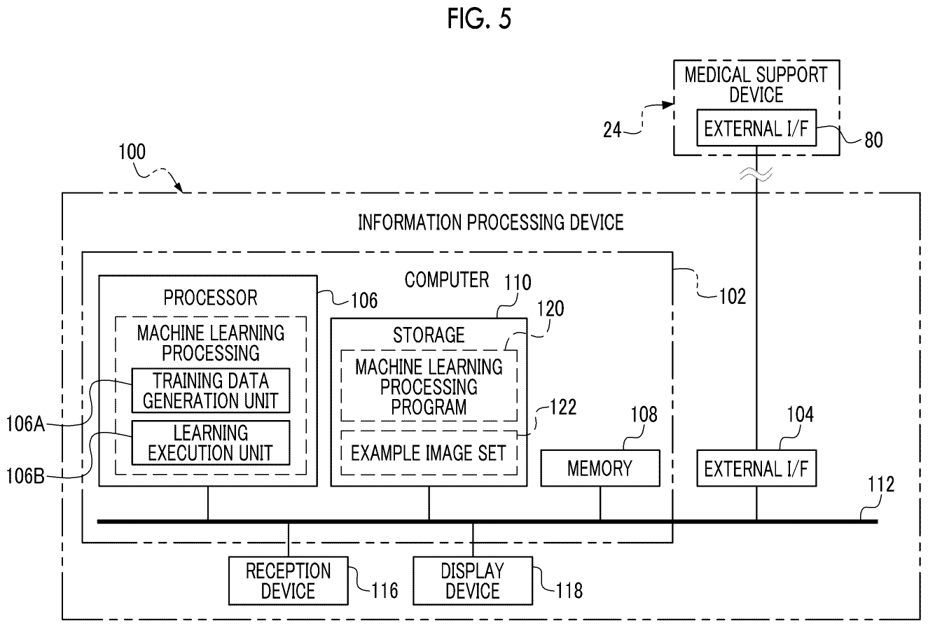

FIG. 5 is a block diagram showing an example of a hardware configuration of an electrical system of an information processing device 100 used for generating the lumen recognition model 92. As shown in FIG. 5, the information processing device 100 comprises a computer 102 and an external I/F 104. The computer 102 comprises a processor 106, a memory 108, and a storage 110. The processor 106, the memory 108, the storage 110, and the external I/F 104 are connected to a bus 112.

It should be noted that a hardware configuration (that is, the processor 106, the memory 108, and the storage 110) of the computer 102 is essentially the same as the hardware configuration of the computer 66, and thus the description of the hardware configuration of the computer 102 will be omitted here.

The information processing device 100 comprises a reception device 116. The reception device 116 is, for example, a keyboard and/or a mouse, and receives an instruction from a user of the information processing device 100 and the like. The reception device 116 is connected to the bus 112. The processor 106 acquires the instruction received by the reception device 116 and operates in accordance with the acquired instruction.

A display device 118 displays various types of information including the image. Examples of the display device 118 include a liquid-crystal display and an EL display. The display device 118 is connected to the bus 112. The processor 106 displays the results obtained by executing various types of processing on the display device 118.

The external I/F 104 transmits and receives various types of information between one or more devices (hereinafter, also referred to as “third external devices”) existing outside the information processing device 100 and the processor 106. The medical support device 24 is connected to the external I/F 104 as one of the third external devices. In the example shown in FIG. 5, the external I/F 80 of the medical support device 24 is connected to the external I/F 104. The external I/F 104 controls the transmission and reception of various types of information between the processor 82 (see FIGS. 3 and 4) of the medical support device 24 and the processor 106 of the information processing device 100. For example, the information processing device 100 generates the lumen recognition model 92, and transmits the generated lumen recognition model 92 to the medical support device 24 via the external I/Fs 80 and 104 in response to a request from the medical support device 24.

A machine learning processing program 120 is stored in the storage 110. The processor 106 reads out the machine learning processing program 120 from the storage 110, and executes the readout machine learning processing program 120 on the memory 108 to execute machine learning processing. The machine learning processing is implemented by the processor 106 operating as a training data generation unit 106A and a learning execution unit 106B in accordance with the machine learning processing program 120 executed on the memory 108.

An example image set 122 is stored in the storage 110. As will be described in detail below, the example image set 122 is used by the training data generation unit 106A.

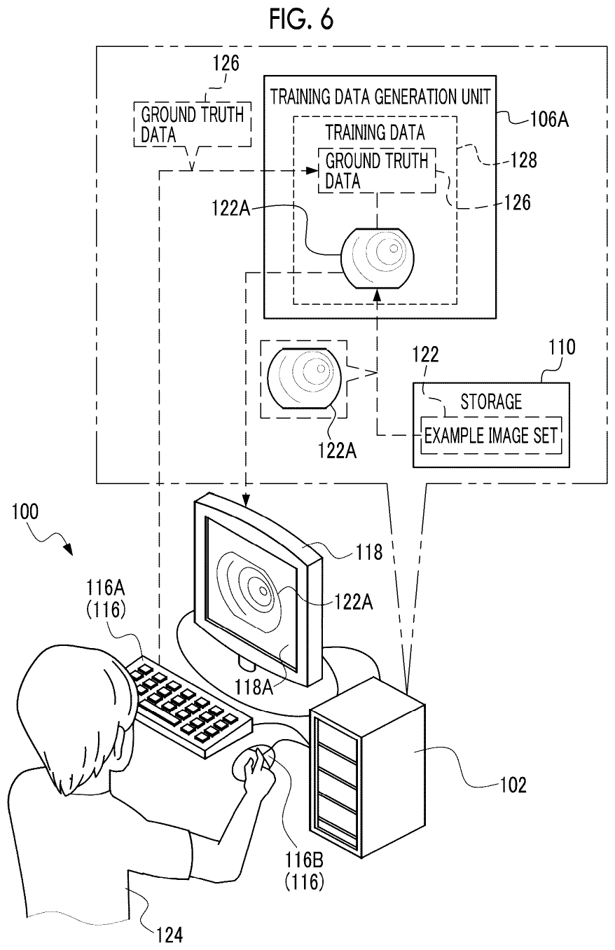

FIG. 6 is a conceptual diagram showing an example of processing contents of the training data generation unit 106A. As shown in FIG. 6, the information processing device 100 is used by an annotator 124. The annotator 124 means an operator who adds annotations for machine learning to given data (that is, an operator who performs labeling).

In the example shown in FIG. 6, a keyboard 116A and a mouse 116B are shown as examples of the reception device 116. The annotator 124 issues an instruction to the computer 102 via the keyboard 116A and the mouse 116B.

The example image set 122 includes a plurality of example images 122A showing different contents. The example image 122A is an image determined in advance as a medical image to be used for object recognition processing (for example, processing in which the recognition unit 82A recognizes the lumen 42 based on the frame 40 and the lumen recognition model 92). The image determined in advance as the medical image to be used for the object recognition processing is an image corresponding to the frame 40. In other words, the image corresponding to the frame 40 can also be said to be an image that represents the frame 40. In other words, the image that represents the frame 40 can also be said to be an image showing a sample of the frame 40. Here, a first example of the image showing the sample of the frame 40 is an image obtained by actually imaging the inside of the large intestine with the camera. A second example of the image showing the sample of the frame 40 is a virtually created image (for example, an image generated by generative AI, such as Stable Diffusion or Midjourney).

The training data generation unit 106A acquires the example image 122A from the example image set 122 in response to the instruction received by the reception device 116. The training data generation unit 106A displays the example image 122A on a screen 118A of the display device 118. In a state in which the example image 122A is displayed on the screen 118A, the annotator 124 indicates a lumen correspondence position, which is the position of the lumen shown in the example image 122A in the example image 122A, with respect to the training data generation unit 106A via the reception device 116. The training data generation unit 106A associates ground truth data 126 with the example image 122A based on the lumen correspondence position indicated via the reception device 116, to generate training data 128. The association of the ground truth data 126 with the example image 122A is implemented by adding an annotation for specifying the lumen correspondence position as the ground truth data 126 to the lumen correspondence position in the example image 122A.

In this way, the training data generation unit 106A repeatedly executes the processing of associating the ground truth data 126 with each of the example images 122A included in the example image set 122 in response to the instruction issued from the annotator 124, to generate a plurality of pieces of training data 128.

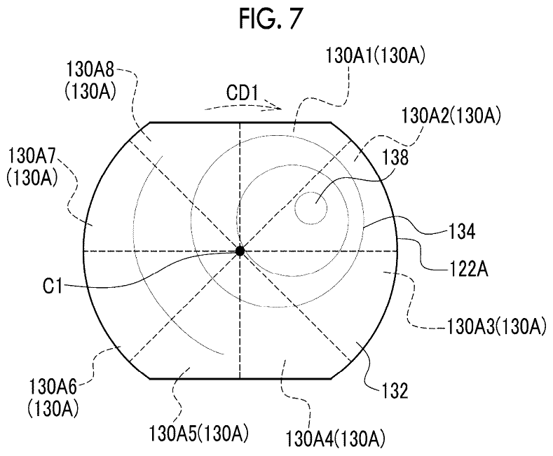

FIG. 7 is a conceptual diagram showing an example of a composition of the example image 122A. As shown in FIG. 7, a large intestine 132 is shown in the example image 122A. In the example shown in FIG. 7, an intestinal wall 136 in which a plurality of folds 134 are formed and a lumen 138 are shown in the example image 122A.

The example image 122A is divided into a plurality of divided regions 130A. Eight divided regions 130A1 to 130A8 are included in the plurality of divided regions 130A. The divided regions 130A1 to 130A8 are regions that radially exist from a center C1 of the example image 122A toward an outer edge of the example image 122A, and are disposed along a circumferential direction CD1 (in other words, around the center C1) of the example image 122A.

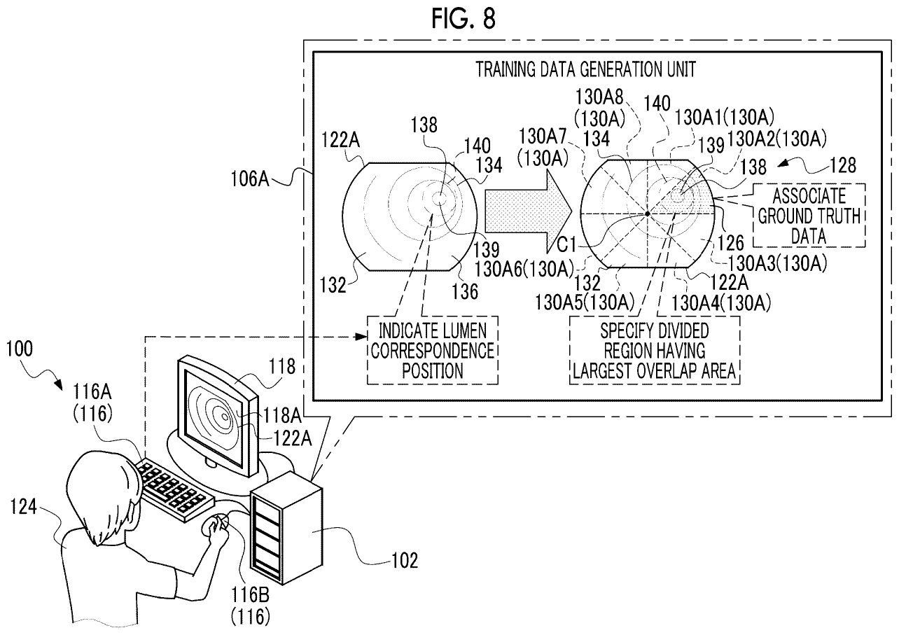

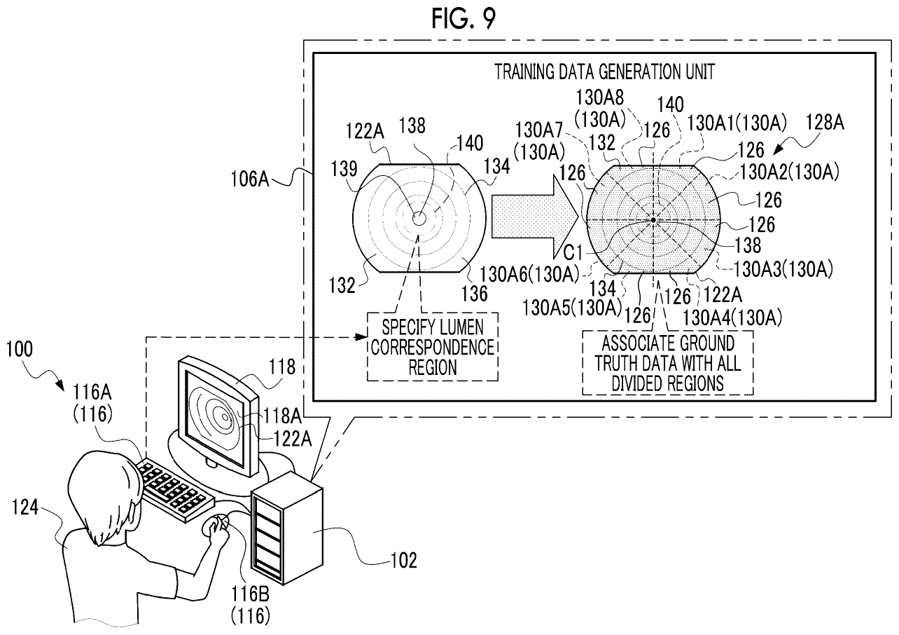

FIGS. 8 and 9 are conceptual diagrams showing an example of a method in which the training data generation unit 106A associates the ground truth data 126 with the example image 122A to generate the training data 128.

As shown in FIG. 8, in a state in which the example image 122A is displayed on the screen 118A, the annotator 124 indicates a lumen correspondence position 139, which is the position of the lumen 138 shown in the example image 122A in the example image 122A, with respect to the training data generation unit 106A via the reception device 116. The training data generation unit 106A displays a circular frame 140 in a superimposed manner on the example image 122A in response to the instruction received by the reception device 116, and disposes the frame 140 at a position surrounding the lumen 138 shown in the example image 122A. The frame 140 is a mark that defines the lumen correspondence position 139 in the example image 122A. That is, a position of a region surrounded by the frame 140 in the example image 122A is the lumen correspondence position 139. The size and the position of the frame 140 are freely changed on the screen 118A in response to the instruction received by the reception device 116. Here, the shape of the frame 140 is a circular shape, but the shape may be another shape than the circular shape. The size of the frame 140 can be changed in response to the instruction received by the reception device 116.

The annotator 124 issues a confirmation instruction, which is an instruction to confirm the lumen correspondence position 139, to the training data generation unit 106A via the reception device 116 in a state in which the frame 140 is disposed at the position surrounding the lumen 138. As a result, the training data generation unit 106A confirms the lumen correspondence position 139.

The training data generation unit 106A specifies the divided region 130A having a largest overlap area with the frame 140 that defines the lumen correspondence position 139 among the plurality of divided regions 130A. Then, the training data generation unit 106A generates the training data 128 by associating the ground truth data 126 with the specified divided region 130A (in the example shown in FIG. 8, the divided region 130A2) as the annotation for specifying the divided region 130A in which the lumen 138 is shown.

FIG. 8 shows an example of a method for generating the training data 128 in a case in which the lumen 138 is shown in a region other than the central region in the example image 122A, whereas FIG. 9 shows an example of a method for generating the training data 128 in a case in which the lumen 138 is shown in the central region in the example image 122A. As shown in FIG. 9, in a case in which the lumen 138 is shown in the central region in the example image 122A, the training data generation unit 106A generates the training data 128 by associating the ground truth data 126 with each of all the divided regions 130A (that is, the divided regions 130A1 to 130A8).

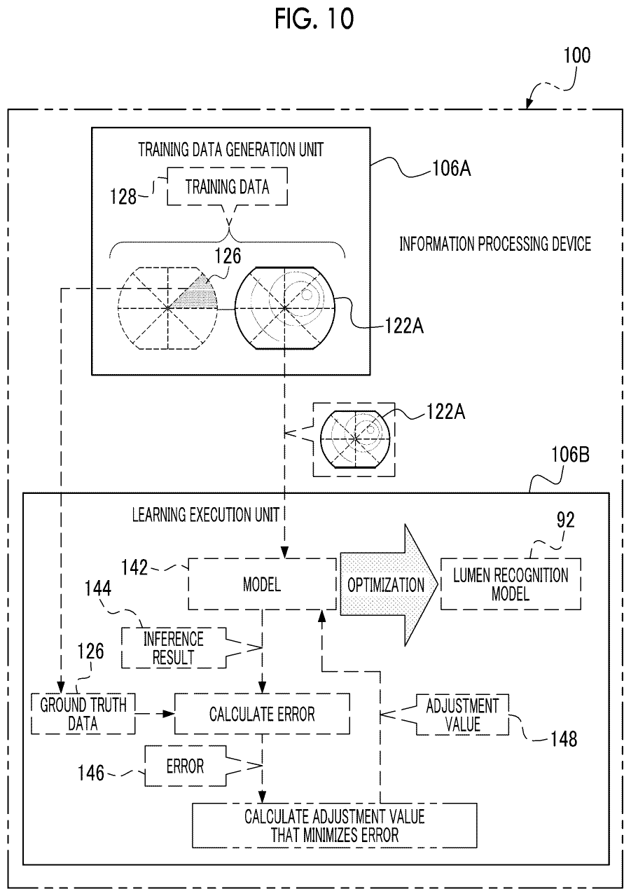

FIG. 10 is a conceptual diagram showing an aspect example in which the learning execution unit 106B executes machine learning using the training data 128 to generate the lumen recognition model 92. As shown in FIG. 10, in the information processing device 100, the learning execution unit 106B acquires the training data 128 generated by the training data generation unit 106A. Then, the learning execution unit 106B executes the machine learning using the training data 128.

In the example shown in FIG. 10, the learning execution unit 106B includes a model 142. Examples of the model 142 include a neural network. Examples of the neural network include a convolutional neural network. The learning execution unit 106B inputs the example image 122A included in the training data 128 to the model 142. In a case in which the example image 122A is input, the model 142 performs an inference to output an inference result 144. The learning execution unit 106B calculates an error 146 between the inference result 144 and the ground truth data 126 included in the training data 128.

The learning execution unit 106B calculates a plurality of adjustment values 148 for minimizing the error 146. Then, the learning execution unit 106B adjusts a plurality of optimization variables in the model 142 by using the plurality of adjustment values 148, to optimize the model 142. For example, the plurality of optimization variables mean a plurality of coupling weights and a plurality of offset values included in the model 142.

The learning execution unit 106B repeatedly executes learning processing of inputting the example image 122A to the model 142, calculating the error 146, calculating the plurality of adjustment values 148, and adjusting the plurality of optimization variables in the model 142 using the plurality of pieces of training data 128. That is, the learning execution unit 106B adjusts the plurality of optimization variables in the model 142 using the plurality of adjustment values 148 calculated such that the error 146 is minimized for each of the plurality of example images 122A included in the plurality of pieces of training data 128, to optimize the model 142. The lumen recognition model 92 is generated by optimizing the model 142 in this manner. The lumen recognition model 92 is transmitted from the information processing device 100 to the medical support device 24 via the external I/Fs 80 and 104 (see FIG. 5), and is received by the medical support device 24. Then, in the medical support device 24, the lumen recognition model 92 is stored in the storage 86 by the processor 82 (see FIG. 4). The lumen recognition model 92 stored in the storage 86 is used by the recognition unit 82A (see FIG. 4).

In a case in which the lumen recognition model 92 is actually used, the frame 40 is input to the lumen recognition model 92. Then, the lumen recognition model 92 recognizes the lumen 42 (see FIG. 1) shown in the input frame 40. A result of the recognition is displayed on the screen 35. For example, a position at which the lumen 42 in the frame 40 is shown is visualized by displaying the mark or the like in correspondence with any of eight regions (that is, eight regions corresponding to the divided regions 130A1 to 130A8) in the frame 40.

However, even in a case in which the mark or the like is displayed and the region in which the lumen 42 is shown is in a state of being visually specifiable, it may be difficult for the doctor 12 to visually ascertain where the lumen 42 is shown in the region corresponding to the position at which the mark or the like is displayed. For example, the smaller a display size of the lumen 42 is with respect to a display size of the region in which the lumen 42 is shown among the eight regions in the frame 40, the more difficult it is to visually ascertain where the lumen 42 is shown in the region corresponding to the position at which the mark or the like is displayed.

Therefore, in view of such circumstances, in the present embodiment, the medical support processing is executed by the processor 82 of the medical support device 24.

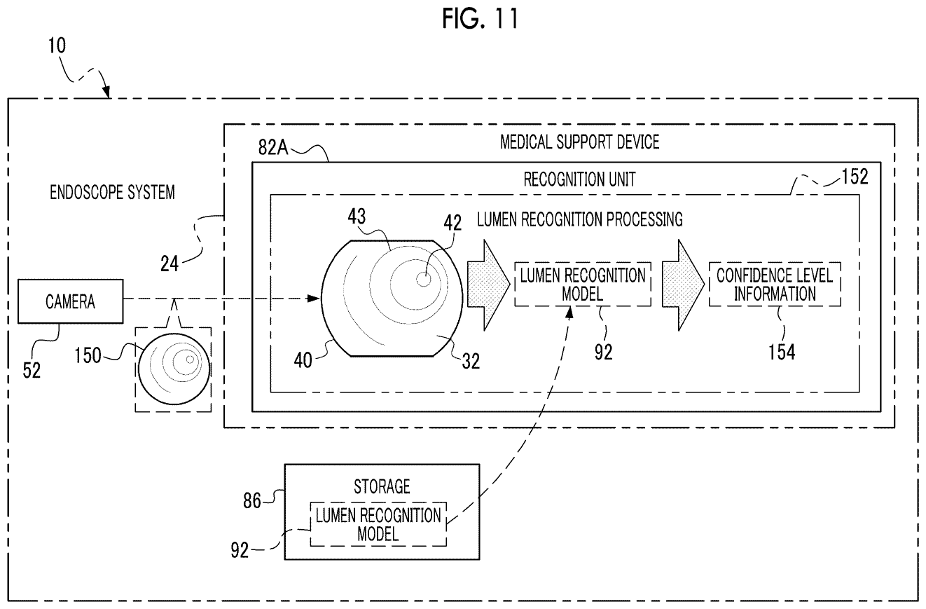

FIG. 11 shows an example of processing contents of the recognition unit 82A. As shown in FIG. 11, an image 150 obtained by imaging the intestinal wall 32 in the large intestine 28 including the lumen 42 with the camera 52 is acquired by the recognition unit 82A. The recognition unit 82A generates the frame 40 by executing various types of processing on the image 150. In the example shown in FIG. 11, the intestinal wall 32 having the folds 43 and the lumen 42 are shown in the frame 40.

The recognition unit 82A executes lumen recognition processing 152 on the frame 40. The lumen recognition processing 152 is processing of recognizing the lumen 42 shown in the frame 40 using the lumen recognition model 92 stored in the storage 86 (in other words, processing of specifying an existence position of the lumen 42, which is shown in the frame 40, in the frame 40 using the lumen recognition model 92). The recognition unit 82A acquires the frame 40 from the camera 52, and inputs the acquired frame 40 to the lumen recognition model 92 to cause the lumen recognition model 92 to generate confidence level information 154.

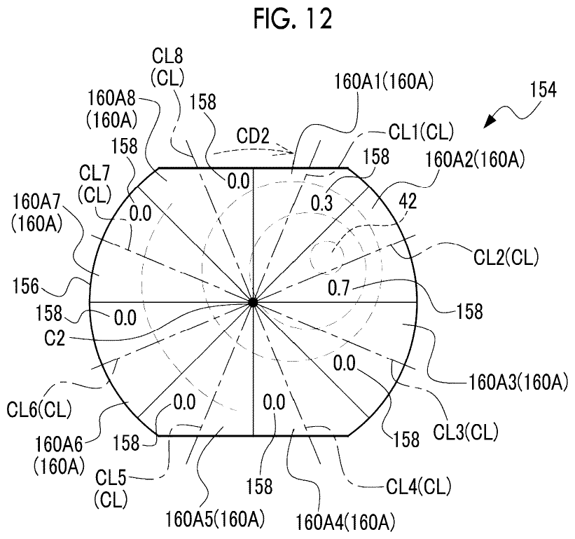

FIG. 12 shows an example of a composition of the confidence level information 154 generated by the lumen recognition model 92 in a case in which the lumen 42 is shown in the frame 40. As shown in FIG. 12, the confidence level information 154 is information including a map 156 corresponding to the frame 40. A size and a shape of the map 156 are the same as a size and a shape of the frame 40. However, this is merely an example, and an outer contour of the map 156 need only be in a similar relationship with an outer contour of the frame 40.

A confidence level 158 (for example, a probability of the existence of the lumen 42) is added to the map 156. Here, the map 156 is shown as an example, but the frame 40 may be used instead of the map 156. The map 156 includes a plurality of divided regions 160A corresponding to the plurality of divided regions 130A (see FIGS. 7 to 9). Each of the plurality of divided regions 160A is a region obtained by dividing the map 156 along a circumferential direction CD2 (in other words, around a center C2 of the map 156). In the example shown in FIG. 12, divided regions 160A1 to 160A8 are shown as examples of the plurality of divided regions 160A. The divided regions 160A1 to 160A8 are regions obtained by dividing the map 156 at intervals of a constant angle (for example, at intervals of 45 degrees) along the circumferential direction CD2. In other words, the divided regions 160A1 to 160A8 can be said to be regions obtained by radially dividing the map 156 into eight regions from the center C2 of the map 156 toward an outer edge of the map 156.

In the present embodiment, the map 156 is an example of an “image corresponding to the medical image” according to the present disclosure. In addition, in the present embodiment, the confidence level 158 is an example of a “confidence level” according to the present disclosure. Further, in the present embodiment, the circumferential direction CD2 is an example of a “circumferential direction” according to the present disclosure, and the divided regions 160A1 to 160A8 are examples of a “plurality of divided regions” according to the present disclosure.

A plurality of center lines CL are provided in the map 156. The plurality of center lines CL correspond to the plurality of divided regions 160A, and are disposed at equal intervals along the circumferential direction CD2. Each of the plurality of center lines CL is a virtual line along a half angle (for example, 22.5 degrees) of the above-described constant angle from the center C2 in each divided region 160A. In the example shown in FIG. 12, center lines CL1 to CL8 are provided as examples of the plurality of center lines CL for the divided regions 160A1 to 160A8. The center lines CL1 to CL8 are disposed around the center C2 at intervals of 45 degrees.

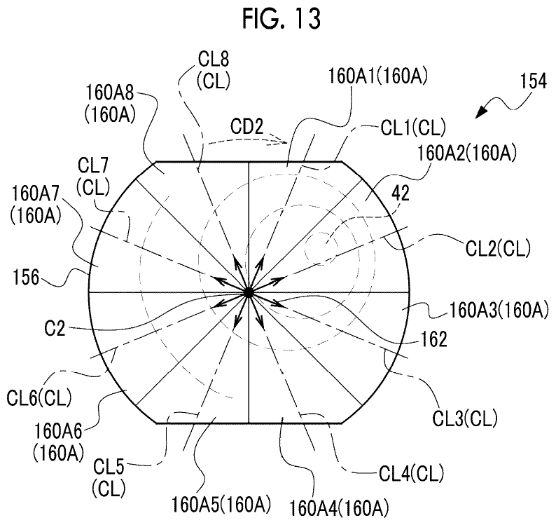

FIG. 13 shows an aspect example in which a plurality of unit direction vectors 162 are added to the map 156. As shown in FIG. 13, a direction from the center C2 to the existence position of each of the plurality of divided regions 160A is determined by the unit direction vector 162. The unit direction vector 162 is added to each of the plurality of divided regions 160A. The unit direction vector 162 is a unit vector indicating a direction from the center C2 to the existence position of the divided region 160 (that is, the existence position of each of the divided regions 160A1 to 160A8). In the example shown in FIG. 13, one unit direction vector 162 is added to each of the plurality of divided regions 160A (that is, the divided regions 160A1 to 160A8), along the center line CL for each divided region 160A. In the present embodiment, the center C2 is an example of a “reference position” according to the present disclosure, and the plurality of unit direction vectors 162 are examples of a “plurality of first vectors” according to the present disclosure.

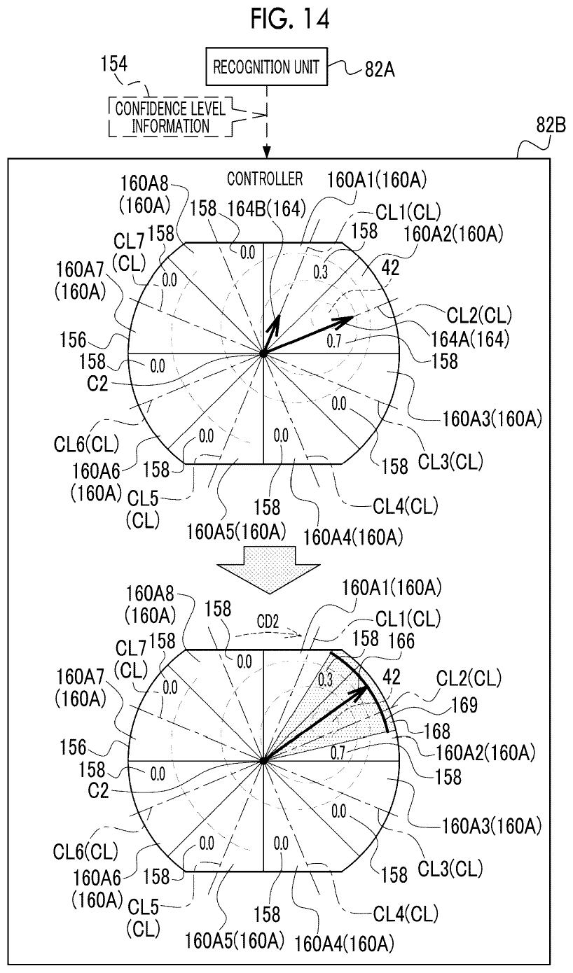

FIG. 14 shows an aspect example in which a plurality of direction vectors 164 are added to the map 156. As shown in FIG. 14, the controller 82B acquires the confidence level information 154 including the map 156 to which the plurality of unit direction vectors 162 (see FIG. 13) are added, from the recognition unit 82A. Then, the controller 82B generates the plurality of direction vectors 164 based on the plurality of unit direction vectors 162 and the plurality of confidence levels 158 included in the confidence level information 154.

The direction vector 164 is a vector of which the magnitude is adjusted by adding, as a weight, the confidence level 158 of the divided region 160A, to which the unit direction vector 162 is added, to the unit direction vector 162. The magnitude of the direction vector 164 corresponds to the height of the confidence level 158, and the direction vector 164 becomes larger as the confidence level 158 becomes higher.

Here, a specific example of a method for generating the direction vector 164 will be described. For example, in the divided region 160A (in the example shown in FIG. 14, the divided region 160A1) to which “0.3” is added as the confidence level 158, a vector obtained by increasing the magnitude of the unit direction vector 162 by 30% is generated as the direction vector 164 (in the example shown in FIG. 14, the direction vector 164B). In addition, for example, in the divided region 160A (in the example shown in FIG. 14, the divided region 160A2) to which “0.7” is added as the confidence level 158, a vector obtained by increasing the magnitude of the unit direction vector 162 by 70% is generated as the direction vector 164 (in the example shown in FIG. 14, the direction vector 164A). In addition, for example, in the divided region 160A (in the example shown in FIG. 14, the divided regions 160A3 to 160A8) to which “0.0” is added as the confidence level 158, the magnitude of the direction vector 164 may be set to “zero”, or the unit direction vector 162 may be used as the direction vector 164 as it is.

It should be noted that the direction vector 164 shown here is merely an example, and a vector obtained by simply multiplying the unit direction vector 162 by the confidence level 158 may be used as the direction vector 164.

In the example shown in FIG. 14, the direction vectors 164A and 164B are shown. The direction vector 164A is a vector obtained by adjusting the magnitude of the unit direction vector 162 added to the divided region 160A2 by the confidence level 158 (0.7 in the example shown in FIG. 14) of the divided region 160A2. The direction vector 164B is a vector obtained by adjusting the magnitude of the unit direction vector 162 added to the divided region 160A2 by the confidence level 158 (0.3 in the example shown in FIG. 14) of the divided region 160A1. The magnitude of the direction vector 164B represents a degree of the possibility of the existence of the lumen 42 in the divided region 160A to which the unit direction vector 162 of the direction vector 164B is added. That is, the larger the direction vector 164B is, the higher the probability of the existence of the lumen 42 in the divided region 160A to which a base unit direction vector 162 of the direction vector 164B is added.

The controller 82B generates a vector sum 166 based on the plurality of direction vectors 164. The vector sum 166 is a sum of the plurality of direction vectors 164. In the example shown in FIG. 14, as the vector sum 166, a vector sum of the direction vector 164A and the direction vector 164B is shown.

A direction from the center C2 to a lumen existence region 168 in the map 156 is determined by the vector sum 166. The lumen existence region 168 refers to a region in which the lumen 42 is shown in the frame 40. The divided region 160A is a region in which the position in the map 156 is constrained, whereas the lumen existence region 168 is a region in which the position is changed depending on the position at which the vector sum 166 is created without the position being constrained in the map 156, as in the divided region 160A. In addition, in the divided region 160A, even in a case in which the lumen 42 exists in the divided region 160A, it is difficult to estimate the existence position of the lumen 42 in the divided region 160A, but, in the lumen existence region 168, since the lumen 42 exists on the line along the vector sum 166, it is easy to estimate the existence position of the lumen 42. Therefore, in the lumen existence region 168, the existence position of the lumen 42 is specified with higher accuracy than in a case in which the existence position of the lumen 42 is specified in the divided region 160A. In other words, the lumen existence region 168 can be said to be a region in which the position at which the lumen 42 is shown in the frame 40 (that is, the existence position of the lumen 42 in the frame 40) can be specified with a higher resolution than in the plurality of divided regions 160A, along the circumferential direction CD2.

The controller 82B specifies the lumen existence region 168 based on the vector sum 166. For example, the controller 82B specifies, as the lumen existence region 168, a region of ±α degrees along the circumferential direction CD2 with one point (for example, an end point) other than a start point of the vector sum 166 as the center. Examples of the ±α degree include ±22.5 degrees. It should be noted that ±22.5 degrees is merely an example, and the range may be narrower or wider than ±22.5 degrees. In addition, the α degree may be a fixed value or a variable value that is changed in accordance with an instruction received by the reception device 64 or various conditions (for example, a type of an operation mode of the endoscope system 10).

The controller 82B generates a mark 169 for specifying the position of the lumen existence region 168 in the map 156, based on the plurality of divided regions 160A and the plurality of confidence levels 158. The mark 169 is visible information determined based on the vector sum 166 generated based on the plurality of divided regions 160A and the plurality of confidence levels 158. In the example shown in FIG. 14, the shape of the mark 169 is an arc in which the end point of the vector sum 166 is a midpoint. A center of the arc, which is the shape of the mark 169, is the center C2 of the map 156. The mark 169 indicates a range from one end to the other end of the lumen existence region 168 in the circumferential direction CD2.

In the present embodiment, the vector sum 166 is an example of a “second vector” according to the present disclosure. Further, in the present embodiment, the direction vector 164 is an example of a “third vector” according to the present disclosure. Further, in the present embodiment, the mark 169 is an example of a “mark” according to the present disclosure.

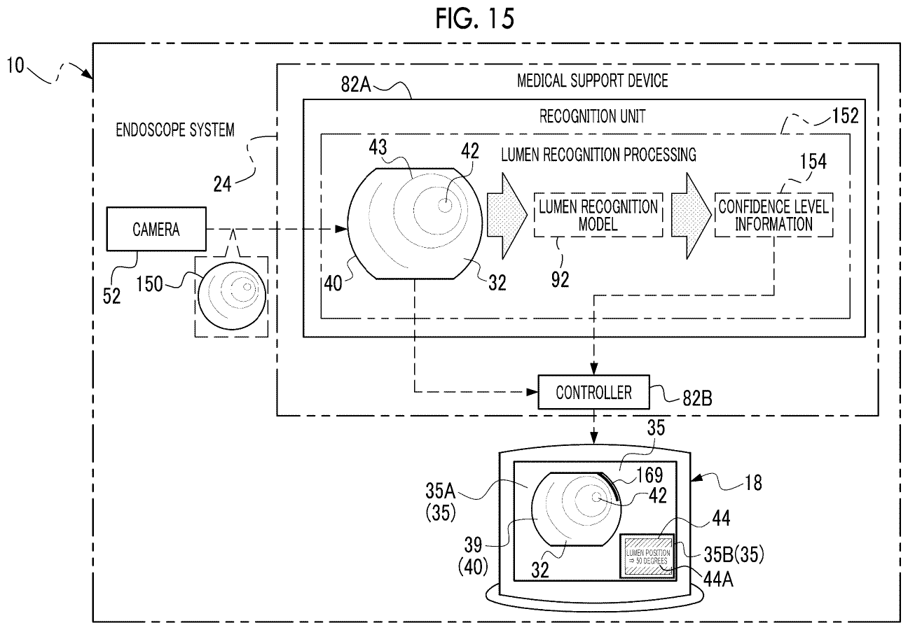

FIG. 15 shows a form example in which the frame 40 and the like are displayed on the screen 35 in a case in which the lumen 42 is shown in a region other than the central region of the frame 40. As shown in FIG. 15, the controller 82B acquires the frame 40 input to the lumen recognition model 92 from the recognition unit 82A in order to obtain the confidence level information 154 including the map 156 used for generating the mark 169. The controller 82B displays the frame 40 acquired from the recognition unit 82A in the first display region 35A, and displays the mark 169 in the first display region 35A in a comparable manner with the frame 40. For example, the mark 169 is displayed in a superimposed manner on the frame 40.

In addition, the controller 82B updates the mark 169 in accordance with a display timing of the frame 40. For example, the controller 82B generates the mark 169 based on the confidence level information 154 to display the mark 169 in a superimposed manner on the frame 40 each time the confidence level information 154 is obtained by the recognition unit 82A. In such a case, the mark 169 displayed in the first display region 35A is updated each time the frame 40 is displayed. It should be noted that the mark 169 displayed in the first display region 35A may be updated on the condition that the frame 40 is updated a plurality of times and displayed in the first display region 35A (for example, the frame 40 is displayed in the first display region 35A in a range of a plurality of sheets to a plurality of hundreds of sheets designated in advance).

In addition, the controller 82B displays visible information 44A in the second display region 35B as one piece of the auxiliary information 44. Examples of the visible information 44A include text for specifying a position of a region corresponding to the lumen existence region 168 in the frame 40, that is, text for specifying a position of the mark 169 displayed in the first display region 35A (for example, text representing an angle indicating a position of the vector sum 166 in a case in which a boundary line between the divided region 160A1 and the divided region 160A8 is 0 degrees). In the present embodiment, the visible information 44A is an example of “lumen specification information” according to the present disclosure.

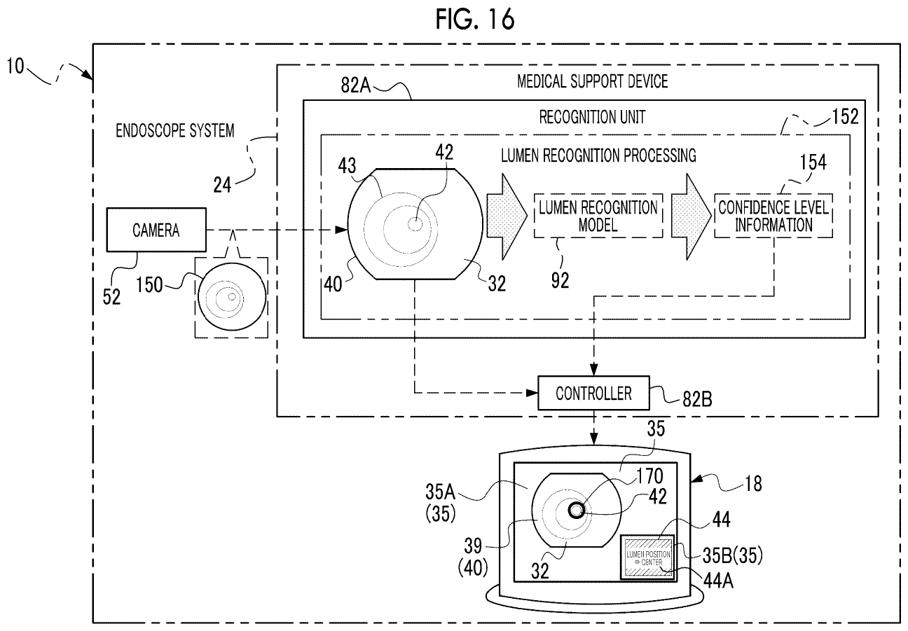

FIG. 16 shows a form example in which the frame 40 and the like are displayed on the screen 35 in a case in which the lumen 42 is shown in the central region of the frame 40 (for example, in a case in which the center of the lumen 42 coincides with the center of the frame 40). As shown in FIG. 16, in a case in which the center of the lumen 42 coincides with the center of the frame 40, the controller 82B displays the frame 40 in the first display region 35A, and displays a mark 170 in a superimposed manner on the frame 40. The mark 170 is a mark (for example, an annular mark) surrounding the lumen 42 shown in the frame 40. In a case in which the direction vectors 164 of all the divided regions 160A are equivalent, that is, in a case in which the vector sum 166 is zero, the mark 170 is generated by the controller 82B and is displayed in a superimposed manner on the frame 40. Further, in this case, the controller 82B displays, as the visible information 44A, information (for example, text) indicating that the lumen 42 is shown at the center of the frame 40 in the second display region 35B.

It should be noted that, here, although the form example has been described in which the mark 170 is generated and is displayed in a superimposed manner on the frame 40 in a case in which the vector sum 166 is zero, this is merely an example. For example, in a case in which the magnitude of the vector sum 166 is less than a threshold value (for example, the magnitude of the unit direction vector 162), the mark 170 may be generated and may be displayed in a superimposed manner on the frame 40. In addition, here, as an example of the mark 170, the mark surrounding the lumen 42 shown in the frame 40 has been described, but this is merely an example, and a mark (for example, a dot located at the center of the lumen 42 shown in the frame 40 or an arrow indicating the position of the lumen 42) for specifying the position of the lumen 42 shown in the frame 40 may be used.

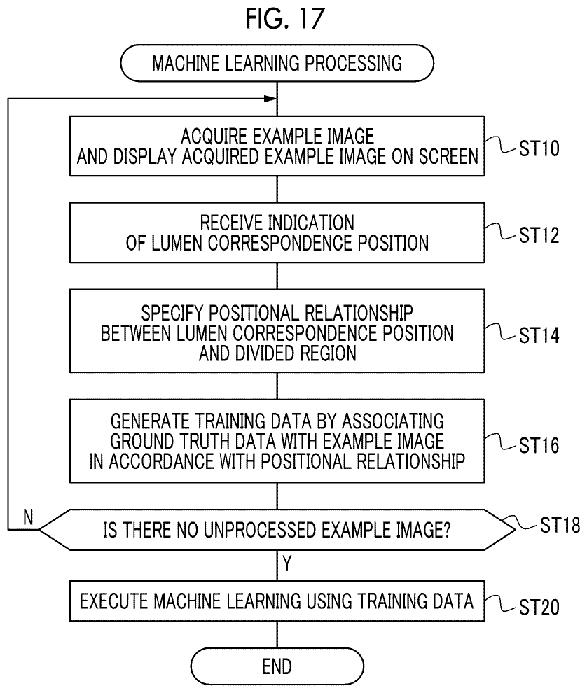

Hereinafter, an operation of the information processing device 100 will be described with reference to FIG. 17.

In the machine learning processing shown in FIG. 17, first, in step ST10, the training data generation unit 106A acquires an unprocessed example image 122A from the example image set 122 stored in the storage 110. Here, the unprocessed example image 122A means the example image 122A that has not yet been used for the machine learning processing. The training data generation unit 106A displays the example image 122A acquired from the example image set 122, on the screen 118A. After the processing of step ST10 is executed, the machine learning processing proceeds to step ST12.

In step ST12, the training data generation unit 106A receives the indication of the lumen correspondence position 139. After the processing of step ST12 is executed, the machine learning processing proceeds to step ST14.

In step ST14, the training data generation unit 106A specifies a positional relationship between the lumen correspondence position 139 received in step ST12 and the plurality of divided regions 130A. After the processing of step ST14 is executed, the machine learning processing proceeds to step ST16.

In step ST16, the training data generation unit 106A associates the ground truth data 126 with the example image 122A acquired in step ST10 in accordance with the positional relationship specified in step ST14. For example, in a case in which the lumen correspondence position 139 exists in a region other than the central region of the example image 122A, the ground truth data 126 is associated with the divided region 130A having the largest overlap area with the lumen correspondence position 139 in response to the instruction issued from the annotator 124. In addition, for example, in a case in which the lumen correspondence position 139 exists in the central region of the example image 122A, the ground truth data 126 is associated with each of the divided regions 130A in response to the instruction issued from the annotator 124. As described above, the training data generation unit 106A associates the ground truth data 126 with the example image 122A to generate the training data 128. The training data 128 generated in this manner is stored in a predetermined storage medium (for example, the storage 110). After the processing of step ST16 is executed, the machine learning processing proceeds to step ST18.

In step ST18, the training data generation unit 106A determines whether or not the unprocessed example image 122A exists. In step ST18, in a case in which the unprocessed example image 122A exists, a negative determination is made, and the machine learning processing proceeds to step ST10. In step ST18, in a case in which the unprocessed example image 122A does not exist, an affirmative determination is made, and the machine learning processing proceeds to step ST20.

In step ST20, the learning execution unit 106B executes machine learning using the plurality of pieces of training data 128 obtained by repeatedly executing the processing in steps ST10 to ST18 to generate the lumen recognition model 92 (see FIG. 10). The lumen recognition model 92 is stored in the storage 86 of the medical support device 24 (see FIG. 4). After the processing of step ST20 is executed, the machine learning processing ends.

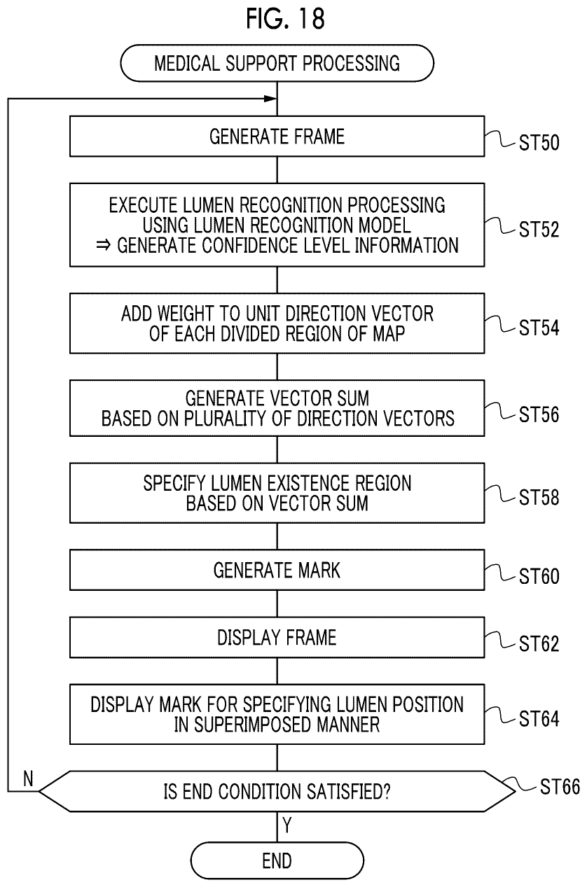

Hereinafter, an operation of a part of the endoscope system 10 according to the present disclosure will be described with reference to FIG. 18. A flow of the medical support processing shown in FIG. 18 is an example of a “medical support method” according to the present disclosure. It should be noted that, hereinafter, for convenience, the description will be made on the premise that the lumen recognition model 92 is stored in the storage 86.

In the medical support processing shown in FIG. 18, in step ST50, the recognition unit 82A acquires the image 150 from the camera 52 and executes various types of processing on the acquired image 150, to generate the frame 40. After the processing of step ST50 is executed, the medical support processing proceeds to step ST52.

In step ST52, the recognition unit 82A executes the lumen recognition processing 152 using the lumen recognition model 92 stored in the storage 86 on the frame 40 generated in step ST50, to generate the confidence level information 154. After the processing of step ST52 is executed, the medical support processing proceeds to step ST54.

In step ST54, the controller 82B generates the plurality of direction vectors 164 by adding, as a weight, the confidence level 158 of the divided region 160A, to which each unit direction vector 162 is added, to the unit direction vector 162 of each divided region 160A of the map 156 included in the confidence level information 154 generated in step ST52. After the processing of step ST54 is executed, the medical support processing proceeds to step ST56.

In step ST56, the controller 82B generates the vector sum 166, which is a sum of the plurality of direction vectors 164 generated in step ST54. After the processing of step ST56 is executed, the medical support processing proceeds to step ST58.

In step ST58, the controller 82B specifies the lumen existence region 168 based on the vector sum 166 generated in step ST56. After the processing of step ST58 is executed, the medical support processing proceeds to step ST60.

In step ST60, the controller 82B generates the mark 169 for specifying the position of the lumen existence region 168 specified in step ST58. After the processing of step ST60 is executed, the medical support processing proceeds to step ST62.

In step ST62, the controller 82B displays the frame 40 generated in step ST50 in the first display region 35A. After the processing of step ST62 is executed, the medical support processing proceeds to step ST64.

In step ST64, the controller 82B displays the mark 169 generated in step ST60 in a superimposed manner on the frame 40 displayed in the first display region 35A. After the processing of step ST64 is executed, the medical support processing proceeds to step ST66.

In step ST66, the controller 82B determines whether or not a medical support processing end condition is satisfied. Examples of the medical support processing end condition include a condition that an instruction to end the medical support processing is issued to the endoscope system 10 (for example, a condition that the reception device 64 receives the instruction to end the medical support processing).

In a case in which the medical support processing end condition is not satisfied in step ST66, a negative determination is made, and the medical support processing proceeds to step ST50. In a case in which the medical support processing end condition is satisfied in step ST66, an affirmative determination is made, and the medical support processing ends.

As described above, in the endoscope system 10, the frame 40 in which the intestinal wall 32 and the lumen 42 are shown is input to the lumen recognition model 92, and thus the confidence level information 154 is generated by the lumen recognition model 92. The confidence level information 154 includes the map 156 divided into the plurality of divided regions 160A. The confidence level 158 indicating that the lumen 42 exists is added to each of the plurality of divided regions 160A.

In the endoscope system 10, the lumen existence region 168 is generated based on the plurality of divided regions 160A and the plurality of confidence levels 158. The lumen existence region 168 is a region in which the existence position of the lumen 42 (that is, the position at which the lumen 42 is shown) is specified with higher accuracy than in the divided region 160A in the frame 40. Since the lumen existence region 168 is generated based on the plurality of divided regions 160A and the plurality of confidence levels 158, the position of the lumen existence region 168 is not fixed in the map 156 as in the divided region 160A. In addition, the position of the lumen existence region 168 in the map 156 is finely changed along the circumferential direction CD2 depending on the existence position of the lumen 42. This means that the lumen existence region 168 is a region in which the existence position of the lumen 42 is defined with a higher resolution than in the plurality of divided regions 160A, along the circumferential direction CD2.

In the endoscope system 10, the mark 169 is generated as information for specifying the lumen existence region 168. Then, the frame 40 input to the lumen recognition model 92 for generating the confidence level information 154 is displayed in the first display region 35A of the screen 35. Further, the mark 169 is displayed in a superimposed manner on the frame 40. This means that the mark 169 is represented on the frame 40 with a higher resolution than in a case in which the confidence level 158 of each of the plurality of divided regions 160A is simply displayed in a superimposed manner on the frame 40 or the visible information (for example, the mark) indicating the height of the confidence level 158 is simply displayed in the first display region 35A.

Therefore, the doctor 12 can more accurately ascertain the position of the lumen 42, which is shown in the frame 40, in the frame 40 by simply visually recognizing the mark 169 displayed in a superimposed manner on the frame 40, as compared with a case in which the confidence level 158 of each of the plurality of divided regions 160A is simply displayed in a superimposed manner on the frame 40 or the visible information indicating the height of the confidence level 158 is simply displayed in the first display region 35A.

In addition, in the endoscope system 10, the mark 169 is displayed in a comparable manner with the frame 40. That is, the mark 169 is displayed in a superimposed manner on the frame 40. As a result, the doctor 12 can visually ascertain the positional relationship between the frame 40 and the mark 169.

In addition, in the endoscope system 10, the direction from the center C2 of the map 156 to the existence position of each of the plurality of divided regions 160A is determined by the plurality of unit direction vectors 162. In addition, the direction from the center C2 of the map 156 to the lumen existence region 168 is determined by the vector sum 166. The vector sum 166 is a sum of the plurality of direction vectors 164 obtained by adding, as the weight, the confidence level 158 to the plurality of unit direction vectors 162. The mark 169, which is displayed in a superimposed manner on the frame 40, is generated based on the vector sum 166.

Here, the plurality of direction vectors 164 are changed in accordance with the confidence level 158 added to each of the plurality of divided regions 160A. The vector sum 166 is changed in accordance with the plurality of direction vectors 164. It can be said that the vector sum 166 is a vector indicating the direction from the center C2 of the map 156 to the existence position of the lumen 42. It can be said that the mark 169 generated based on the vector sum 166 is visible information that represents the existence position of the lumen 42, which is shown in the frame 40, in the frame 40 with higher accuracy as compared with a case in which the confidence level 158 of each of the plurality of divided regions 160A is simply displayed in a superimposed manner on the frame 40 or the visible information indicating the height of the confidence level 158 is simply displayed in the first display region 35A.

Therefore, the doctor 12 can more accurately ascertain the position of the lumen 42, which is shown in the frame 40, in the frame 40 by simply visually recognizing the mark 169 displayed in a superimposed manner on the frame 40, as compared with a case in which the confidence level 158 of each of the plurality of divided regions 160A is simply displayed in a superimposed manner on the frame 40 or the visible information indicating the height of the confidence level 158 is simply displayed in the first display region 35A.

Further, in the endoscope system 10, the mark 169 and/or 170 displayed in the first display region 35A is updated in accordance with the display timing of the frame 40. Further, the visible information 44A displayed in the second display region 35B is also updated in accordance with the display timing of the frame 40. Therefore, the doctor 12 can visually recognize the mark 169 and the visible information 44A (that is, the mark 169 and the visible information 44A for specifying the position of the lumen 42, which is shown in the frame 40, in the frame 40 displayed in the first display region 35A) in accordance with the contents of the frame 40 displayed in the first display region 35A.

In addition, in the endoscope system 10, the shape of the mark 169 is the arc, and the center of the arc is the center of the frame 40. The mark 169 is not superimposed on the entire frame 40, but is superimposed only on a part of the frame 40. By displaying the mark 169 on the frame 40 in a superimposed manner, the doctor 12 can accurately ascertain the position of the lumen 42, which is shown in the frame 40, in the frame 40, while ensuring the visibility of the frame 40 displayed in the first display region 35A.

It should be noted that, in the above-described embodiment, the arc is shown as an example of the shape of the mark 169, but this is merely an example, and the mark may have another shape.

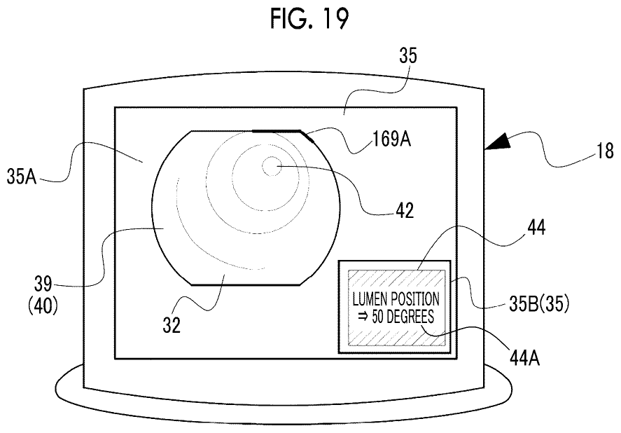

As shown in FIG. 19 as an example, a mark 169A having a shape along the outer edge of the frame 40 may be generated and displayed in the first display region 35A instead of the mark 169. Examples of a method for generating the mark 169A include a method in which the mark 169 is projected to the outer edge of the map 156 from the center C2 side to generate the mark having a shape along the outer edge of the map 156 as the mark 169A. The frame 40 input to the lumen recognition model 92 for generating the confidence level information 154 including the map 156 is displayed in the first display region 35A, and the mark 169A is displayed in accordance with the display timing of the frame 40.

Here, since the mark 169 is formed inside the map 156, as an example of the method for generating the mark 169A, the form example has been described in which the mark 169 is projected to the outer edge of the map 156 from the center C2 side, but, in a case in which the mark 169 is formed outside the map 156 (for example, in a case in which the mark 169 is formed below an upper end of the map 156 or below a lower end of the map 156), the mark 169 need only be projected to the outer edge of the map 156 from the outside of the map 156 toward the center C2 side.

In this way, in a case in which the mark having a shape along the outer edge of the frame 40 is displayed in the first display region 35A, the display of the object that visually blocks the frame 40 in the first display region 35A is suppressed, so that it is possible to allow the doctor 12 to accurately ascertain the position of the lumen 42, which is shown in the frame 40, in the frame 40, while ensuring the visibility of the frame 40 displayed in the first display region 35A.

Further, the mark 169A may also be updated in accordance with the display timing of the frame 40 in the same manner as the mark 169 is updated in accordance with the display timing of the frame 40. In this way, the doctor 12 can visually recognize the mark 169A in accordance with the contents of the frame 40 displayed in the first display region 35A.

In the above-described embodiment, the form example has been described in which the mark 169 is displayed in a superimposed manner on the frame 40, but this is merely an example, and the mark 169 may be displayed outside the frame 40. In a case in which the mark 169 is displayed outside the frame 40, there is no object that visually blocks the frame 40 displayed in the first display region 35A, and thus the visibility of the frame 40 displayed in the first display region 35A can be improved.

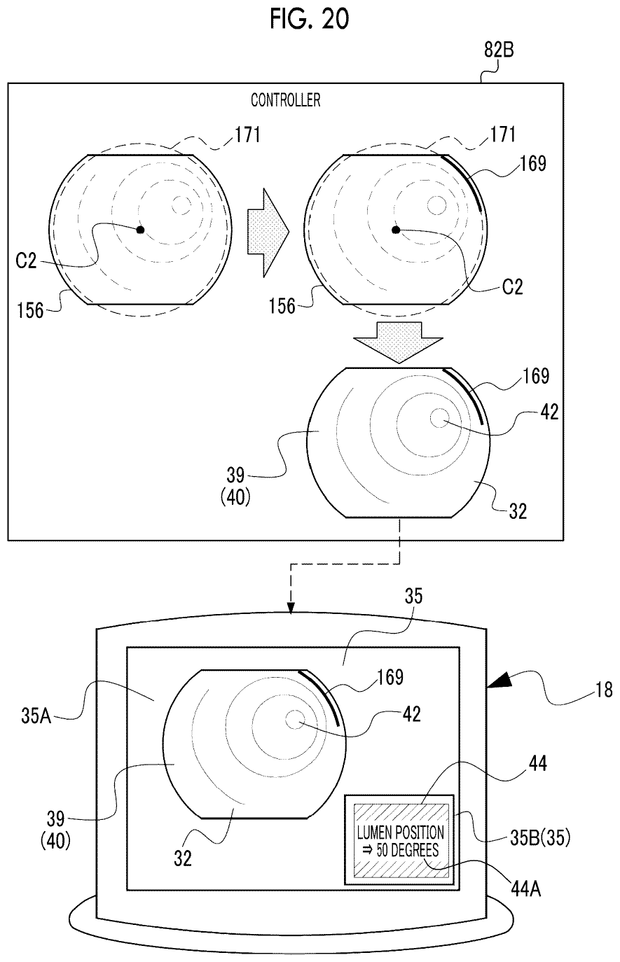

In the above-described embodiment, the form example has been described in which the controller 82B generates the arc-shaped mark 169 for specifying the range from one end to the other end of the lumen existence region 168 in the circumferential direction CD2, and displays the generated mark 169 in the first display region 35A, but this is merely an example. For example, as shown in FIG. 20, the controller 82B may display the mark 169 in the first display region 35A by displaying at least one marker 171 for specifying the range from one end to the other end of the lumen existence region 168 in the circumferential direction CD2 among a plurality of hidden markers 171 associated with the map 156 in the first display region 35A. For example, the number of the plurality of markers 171 (in other words, the number of divided parts) may be “40” (=8×5) obtained by equally dividing each of the eight divided regions 160A (see FIGS. 12 to 14) into five parts or “80” (=8×10) obtained by equally dividing each of the eight divided regions 160A into ten parts. The number of the plurality of markers 171 may be a number other than these numbers. As the number of the markers 171 increases, the resolution of the display of the mark 169 increases. That is, the controller 82B can generate and display the mark 169 in more detail as the number of the markers 171 increases.

In the example shown in FIG. 20, a plurality of arc-shaped markers disposed at constant intervals along a circle having a center that coincides with the center C2 of the map 156 are shown as examples of the plurality of hidden markers 171.

In the example shown in FIG. 20, the display of the mark 169 in the first display region 35A is implemented by displaying at least one marker 171 for specifying the range of the lumen existence region 168 from one end to the other end in the circumferential direction CD2.

In the example shown in FIG. 20, the mark 169 is displayed in a superimposed manner on the frame 40 displayed in the first display region 35A, but the mark 169 may be displayed outside the frame 40 depending on the position of the lumen existence region 168. For example, at least one marker 171 at a position (in the example shown in FIG. 20, above the upper end of the map 156 in a front view and below the lower end of the map 156 in a front view) away from the map 156 is displayed, and thus the mark 169 is displayed outside the frame 40 displayed in the first display region 35A.

In the example shown in FIG. 20, the plurality of arc-shaped markers disposed at constant intervals along a circle having a center that coincides with the center C2 of the map 156 have been shown as examples of the plurality of hidden markers 171, but this is merely an example. For example, the plurality of hidden markers 171 may be a plurality of markers disposed at constant intervals along the outer edge of the map 156.

In the example shown in FIG. 20, the plurality of arc-shaped markers that are disposed at constant intervals along a circle that has a center coinciding with the center C2 of the map 156 and a part of which overlaps the map 156 have been shown, but this is merely an example. For example, a plurality of arc-shaped markers that are disposed at constant intervals along a circle that has a center coinciding with the center C2 of the map 156 and surrounds the map 156 may be used. In this case, the mark 169 is displayed outside the frame 40 by displaying at least one marker 171 for specifying a range from one end to the other end of the lumen existence region 168 in the circumferential direction CD2.

As described above, the display of the mark 169 is implemented by displaying at least one marker 171 at a position corresponding to the position of the lumen existence region 168 among the plurality of hidden markers 171, so that a processing load required for the display of the mark 169 can be reduced.

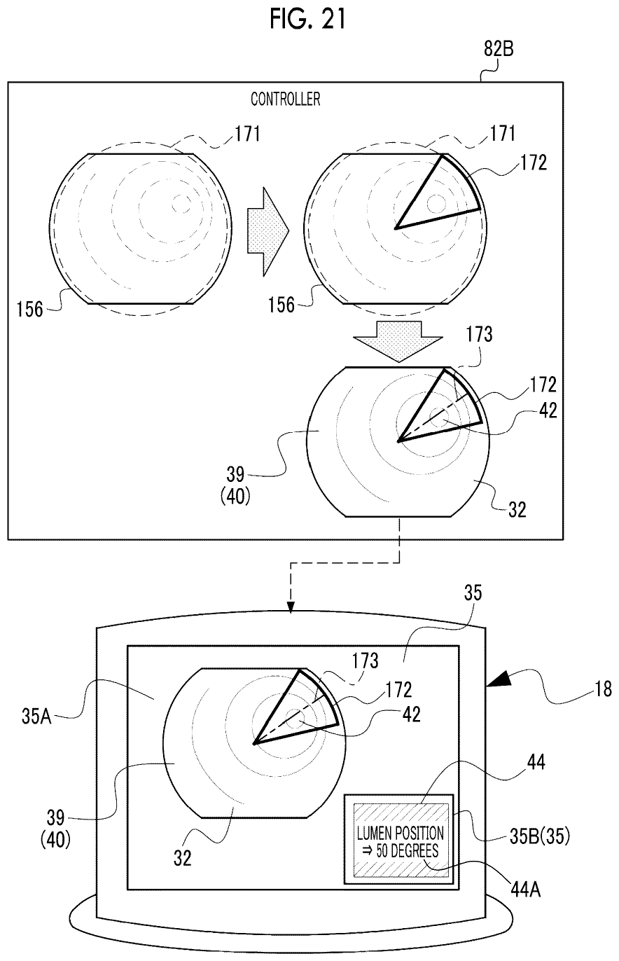

In the example shown in FIG. 20, the form example has been described in which the mark 169 is displayed, but this is merely an example, and, for example, as shown in FIG. 21, an outer contour line 172 may be displayed in the first display region 35A instead of the mark 169. The outer contour line 172 is a line that outlines the outer contour of the lumen existence region 168. In the example shown in FIG. 21, the outer contour line 172 is displayed in a superimposed manner on the frame 40 displayed in the first display region 35A. In a case in which the outer contour line 172 is displayed in a superimposed manner on the frame 40 in this way, the doctor 12 can visually recognize that the lumen 42 is shown within the outer contour line 172.

In addition, as shown in FIG. 21 as an example, a line segment 173 (for example, a line segment extending from the center C2 along the vector sum 166) along the vector sum 166 may be displayed in a superimposed manner on the frame 40. In this case, the doctor 12 can visually recognize that the lumen 42 is shown on the line segment 173 displayed in a superimposed manner on the frame 40.

In the above-described embodiment, eight divided regions 160A are shown as examples, but the number of the divided regions 160A may be less than eight or may be nine or more. In addition, the number of the divided regions 130A need only also be determined in accordance with the number of the divided regions 160A.

In the above-described embodiment, the form example has been described in which the frame 40 is displayed on the screen 35 and the mark 169 is displayed in a comparable manner with the frame 40 (for example, the form example in which the mark 169 is displayed in a superimposed manner on the frame 40), but this is merely an example. For example, the map 156 may be displayed on the screen 35, and the mark 169 may be displayed in a comparable manner with the map 156. One example of the comparable display example is displaying the mark 169 in a superimposed manner on the map 156. It should be noted that, here, the map 156 is an example of an “image corresponding to the medical image” according to the present disclosure.

In the above-described embodiment, the form example has been described in which the visible information 44A is displayed in the second display region 35B, but this is merely an example. For example, audible information (for example, an electronic sound or a language sound) for specifying the position of the lumen 42 in the frame 40 shown in the frame 40 may be output from a speaker (not shown). In addition, the information in which the frame 40 and the mark 169 or the like are combined and/or the visible information 44A may be printed on the medium by a printer. In addition, the information in which the frame 40 and the mark 169 or the like are combined, the visible information 44A, and/or the above-described audible information may be stored in a storage medium (for example, a storage provided in an external device, such as the storage 76, the storage 86, or the server).

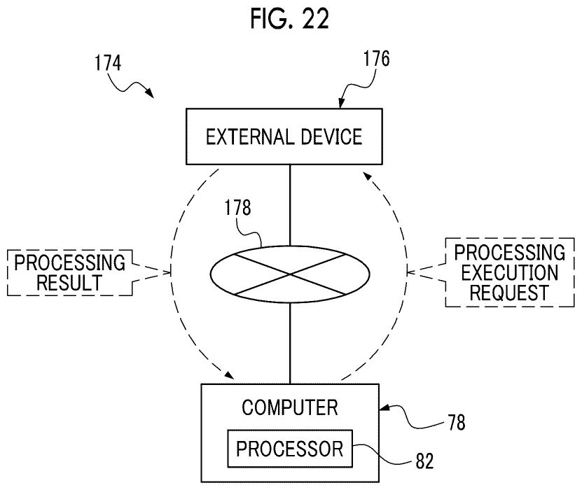

In the above-described embodiment, the form example has been described in which the medical support processing is executed by the computer 78, but the present disclosure is not limited to this, and at least a part of the medical support processing may be executed by a device provided outside the computer 78. Hereinafter, an example of this case will be described with reference to FIG. 22.

FIG. 22 is a conceptual diagram showing an example of a configuration of an endoscope system 174. In the example shown in FIG. 22, the endoscope system 174 is an example of an “endoscope system” according to the present disclosure. The endoscope system 174 is different from the endoscope system 10 according to the above-described embodiment in that an external device 176 is provided.

For example, the external device 176 is a server and is connected to the computer 78 via a network 178 (for example, a WAN and/or a LAN) such that it can communicate with the computer 78. Here, although the server is shown as an example, at least one personal computer or the like may be used as the external device 176 instead of the server.

Examples of the external device 176 include at least one server that directly or indirectly transmits and receives data to and from the computer 78 via the network 178. The external device 176 receives a processing execution instruction issued from the processor 82 of the computer 78 via the network 178. Then, the external device 176 executes processing corresponding to the received processing execution instruction, and transmits a processing result to the computer 78 via the network 178. In the computer 78, the processor 82 receives the processing result transmitted from the external device 176 via the network 178, and executes processing using the received processing result.

Examples of the processing execution instruction include an instruction for the external device 176 to execute at least a part of the medical support processing. A first example of the at least a part of the medical support processing (that is, processing to be executed by the external device 176) is the lumen recognition processing 152. In this case, the external device 176 executes the lumen recognition processing 152 in response to the processing execution instruction issued from the processor 82 via the network 178, and transmits information including the confidence level information 154 as a first processing result to the computer 78 via the network 178. In the computer 78, the processor 82 receives the first processing result and executes the same processing as the processing in the above-described embodiment using the received first processing result.

A second example of the at least a part of the medical support processing (that is, the processing to be executed by the external device 176) is the processing via the controller 82B. In this case, the external device 176 executes the processing via the controller 82B in response to the processing execution instruction issued from the processor 82 via the network 178, and transmits a second processing result (for example, the mark 169 and/or the visible information 44A) to the computer 78 via the network 178. In the computer 78, the processor 82 receives the second processing result and executes the same processing (for example, the display using the display device 18) as the processing in the above-described embodiment using the received second processing result.

It should be noted that the external device 176 may be implemented by cloud computing. The cloud computing is merely an example, and the external device 176 may be implemented by network computing, such as fog computing, edge computing, or grid computing.

In the above-described embodiment, the form example has been described in which the medical support program 90 is stored in the storage 86, but the present disclosure is not limited to this. For example, the medical support program 90 may be stored in a portable computer-readable non-transitory storage medium, such as an SSD or a USB flash drive. The medical support program 90, which is stored in the non-transitory storage medium, is installed in the computer 78 of the endoscope system 10. The processor 82 executes the medical support processing in accordance with the medical support program 90.

In addition, the medical support program 90 may be stored in a storage device of another computer, a server, or the like that is connected to the endoscope system 10 via the network, and the medical support program 90 may be downloaded and installed in the computer 78 in response to a request from the endoscope system 10.

It is not necessary to store the entire medical support program 90 in a storage device of another computer or a server device connected to the endoscope system 10 or to store the entire medical support program 90 in the storage 86, and a part of the medical support program 90 may be stored.

The following various processors can be used as hardware resources for executing the medical support processing. An example of the processor is a CPU which is a general-purpose processor that executes software, that is, a program, to function as the hardware resource for executing the medical support processing. In addition, an example of the processor is a dedicated electric circuit which is a processor having a dedicated circuit configuration designed to execute specific processing, such as an FPGA, a PLD, or an ASIC. All processors have a memory built therein or connected thereto, and all processors use the memory to execute the medical support processing.

The hardware resource for executing the medical support processing may be configured by one of the various processors or by a combination of two or more processors of the same type or different types (for example, a combination of a plurality of FPGAs or a combination of a CPU and an FPGA). Further, the hardware resource for executing the medical support processing may be one processor.

A first example of the configuration in which the hardware resource is configured by one processor is an aspect in which one processor is configured by a combination of one or more CPUs and software and functions as the hardware resource for executing the medical support processing. As a second example, as typified by an SoC or the like, there is a form in which a processor that implements all functions of a system including a plurality of hardware resources executing the medical support processing with one IC chip is used. As described above, the medical support processing is implemented by using one or more of the various processors as the hardware resource.

Further, as the hardware structure of the various processors, specifically, an electronic circuit in which circuit elements, such as semiconductor elements, are combined can be used. Further, the above-described medical support processing is merely an example. Therefore, it goes without saying that unnecessary steps may be deleted, new steps may be added, or the processing order may be changed, within a range that does not deviate from the gist of the present disclosure.

The above-described contents and the above-shown contents are the detailed description of the parts according to the present disclosure, and are merely examples of the present disclosure. For example, the descriptions of the configurations, the functions, the operations, and the effects are the descriptions of the examples of the configurations, the functions, the operations, and the effects of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. Further, in order to avoid confusion and to facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which each of the documents, the patent applications, and the technical standards are specifically and individually stated to be described by reference.

What is claimed is:

1. A medical support device comprising:
a processor,
wherein the processor is configured to:
input a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and
output lumen specification information for specifying a lumen existence region in which an existence position of the lumen is specified with higher accuracy than in the divided regions in the medical image or the image corresponding to the medical image, based on the plurality of divided regions and the plurality of confidence levels.

2. The medical support device according to claim 1,
wherein the lumen existence region is a region in which a position at which the lumen is shown in the medical image is specifiable with a higher resolution than in the plurality of divided regions, along the circumferential direction.

3. The medical support device according to claim 1,
wherein a direction from a reference position of the medical image or the image corresponding to the medical image to an existence position of each of the plurality of divided regions is determined by a plurality of first vectors,
a direction from the reference position to the lumen existence region is determined by a second vector,
the second vector is a sum of at least two third vectors obtained by adding the confidence level, as a weight, to at least two first vectors among the plurality of first vectors, and
the lumen specification information is information determined based on the second vector.

4. The medical support device according to claim 1,
wherein the processor is configured to output the medical image or the image corresponding to the medical image, and
the lumen specification information is updated in accordance with an output timing of the medical image or the image corresponding to the medical image.

5. The medical support device according to claim 1,
wherein the output of the lumen specification information is implemented by displaying the lumen specification information on a screen.

6. The medical support device according to claim 5,
wherein the medical image or the image corresponding to the medical image is displayed on the screen, and
the lumen specification information displayed on the screen is updated in accordance with a display timing of the medical image or the image corresponding to the medical image.

7. The medical support device according to claim 5,
wherein the medical image and/or the image corresponding to the medical image and the lumen specification information are displayed on the screen in a comparable manner.

8. The medical support device according to claim 7,
wherein the lumen specification information is displayed in a superimposed manner on the medical image and/or the image corresponding to the medical image.

9. The medical support device according to claim 1,
wherein a direction from a reference position of the medical image or the image corresponding to the medical image to an existence position of each of the plurality of divided regions is determined by a plurality of first vectors,
a direction from the reference position to the lumen existence region is determined by a second vector,
the second vector is a sum of at least two third vectors obtained by adding the confidence level, as a weight, to at least two first vectors among the plurality of first vectors, and
the lumen specification information includes a mark for specifying a region determined as the lumen existence region based on the second vector in the medical image or the image corresponding to the medical image.

10. The medical support device according to claim 9,
wherein a shape of the mark is an arc, and
a center of the arc is a center of the medical image or the image corresponding to the medical image.

11. The medical support device according to claim 9,
wherein a shape of the mark is a shape along an outer edge of the medical image or an outer edge of the image corresponding to the medical image.

12. The medical support device according to claim 9,
wherein a plurality of markers that are hidden are associated with the medical image or the image corresponding to the medical image,
the medical image or the image corresponding to the medical image is displayed on a screen, and
the mark is displayed on the screen by displaying at least one marker corresponding to a position of the lumen existence region among the plurality of markers.

13. An endoscope system comprising:
the medical support device according to claim 1; and
an endoscope,
wherein the medical image is generated by imaging the inside of the luminal organ including the lumen with the endoscope.

14. A medical support method comprising:
inputting a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and
outputting lumen specification information for specifying a lumen existence region in which an existence position of the lumen is specified with higher accuracy than in the divided regions in the medical image or the image corresponding to the medical image, based on the plurality of divided regions and the plurality of confidence levels.

15. A non-transitory computer-readable storage medium storing a program executable by a computer to execute a process comprising:
inputting a medical image generated by imaging an inside of a luminal organ including a lumen to a trained model to generate a plurality of confidence levels that correspond to a plurality of divided regions obtained by dividing the medical image or an image corresponding to the medical image along a circumferential direction, and that indicate that the lumen is shown in the plurality of divided regions; and
outputting lumen specification information for specifying a lumen existence region in which an existence position of the lumen is specified with higher accuracy than in the divided regions in the medical image or the image corresponding to the medical image, based on the plurality of divided regions and the plurality of confidence levels.
